(19) United States
(12) Patent Application Publication
(10) Pub. No.: US 2024/0024313 A1
(43) Pub. Date: Jan. 25, 2024

(54) BIARYL ETHER UREA COMPOUNDS

(71) Applicant: Jazz Pharmaceuticals Ireland Limited, Dublin (IE)

(72) Inventors: Ju SHI, Mountain View, CA (US); Sheryl COPPOLA, South Jordan, UT (US); Polly Rae PINE, Dillon, CO (US); Matthew BOYLAN, Dublin (IE); Thomas Wesley STOREY, Thousand Oaks, CA (US)

(21) Appl. No.: 18/353,770

(22) Filed: Jul. 17, 2023

Related U.S. Application Data
(60) Provisional application No. 63/389,763, filed on Jul. 15, 2022.

(51) Int. Cl. A61K 31/501; A61K 31/4545; A61K 45/06

(52) U.S. Cl. CPC A61K 31/501; A61K 31/4545; A61K 45/06

(57) ABSTRACT

The disclosure is directed to compounds of Formula (I) and pharmaceutically acceptable salts and solvates thereof. The disclosure is also directed to pharmaceutical compositions containing compounds of Formula (I) and pharmaceutically acceptable salts or solvates thereof, and uses of the pharmaceutical compositions in treating diseases, conditions, and disorders associated with fatty acid amide hydrolase (FAAH) activity.

### CROSS-REFERENCE TO RELATED APPLICATIONS

This application claims priority to U.S. Provisional Application No. 63/389,763, filed Jul. 15, 2022, all of which are herein incorporated by reference in their entireties.

### FIELD

The present disclosure relates to biaryl ether urea compounds and the pharmaceutically acceptable salts of such compounds. The disclosure also relates to compositions, including pharmaceutical compositions comprising such compounds and uses of such compounds and compositions in treating diseases, conditions, and disorders associated with fatty acid amide hydrolase (FAAH) activity.

### BACKGROUND

Fatty acid amides represent a family of bioactive lipids with diverse cellular and physiological effects. Fatty acid amides are hydrolyzed to their corresponding fatty acids by an enzyme known as fatty acid amide hydrolase (FAAH). FAAH is a mammalian integral membrane serine hydrolase responsible for the hydrolysis of some primary and secondary fatty acid amides, including the neuromodulatory compounds anandamide and oleamide.

Anandamide (arachidonoyl ethanolamide) has been shown to possess cannabinoid-like analgesic properties and is released by stimulated neurons. The effects and endogenous levels of anandamide increase with pain stimulation, implying its role in suppressing pain neurotransmission and behavioral analgesia. FAAH inhibitors that elevate brain anandamide levels have demonstrated efficacy in animal models of pain, inflammation, anxiety, and depression. There are, however, inadequate FAAH inhibitors for use as medicines.

### SUMMARY

In some aspects, provided are compositions comprising biaryl ether urea compounds of Formula (I), including pharmaceutical compositions comprising such compounds, and uses of the compositions (including pharmaceutical compositions) in treating various diseases, conditions, and disorders.

In certain embodiments, provided is a pharmaceutical composition comprising a compound of Formula (I) or a pharmaceutically acceptable salt or solvate thereof, and at least one pharmaceutically acceptable excipient, wherein Formula (I) is:

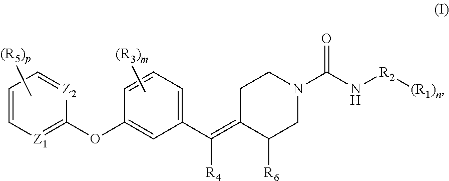

wherein:
each R₁ is independently hydrogen, —C₁-C₆ alkyl, —OH, or —O—(C₁-C₆ alkyl);
R₂ is —C(O)—C₁-C₆ alkyl-C(O)—R₇, a 6-membered aromatic heterocycle containing 1 or 2 nitrogen ring heteroatoms, or dihydropyridazinone, wherein R₇ is hydrogen, halogen, —C₁-C₆ alkyl, —OH, —O—C₁-C₆ alkyl, or —O-haloalkyl;
R₆ is hydrogen or —OH;
each R₃ is independently hydrogen, halogen, —C₁-C₆ alkyl, —(CH₂)₀₋₃—C₃-C₆ cycloalkyl, or —O—C₁-C₆ alkyl;
R₄ is hydrogen, —OH, —C₁-C₆ alkyl, phenyl, or halogen;
each R₅ is independently hydrogen, halogen, haloalkyl, —O-haloalkyl, —C₁-C₆ alkyl, —C(O)C₁-C₆ alkyl, —O—C₁-C₆ alkyl, —S—C₁-C₆ alkyl, —(CH₂)₀₋₃—C₃-C₆ cycloalkyl, CN, aryl, and heteroaryl; wherein the —C₁-C₆ alkyl, —O(C₁-C₆ alkyl), —(CH₂)₀₋₃—C₃-C₆ cycloalkyl, aryl, and heteroaryl groups being optionally independently substituted with from 1 to 4 —C₁-C₆ alkyl, —OH, or halogen substituents;
m is 0, 1, 2, 3, or 4;
n is 0, 1, 2, 3, or 4;
p is 0, 1, 2, 3, or 4; and
Z₁ and Z₂ are independently selected from N and CH,
with the proviso that when R₂ is a 6-membered aromatic heterocycle containing 1 or 2 nitrogen ring heteroatoms, R₆ is not H.

In certain aspects, provided is a method of inhibiting fatty acid amide hydrolase (FAAH) in a subject in need thereof, comprising administering to the subject any of the compounds and compositions, including the pharmaceutical compositions, as described herein.

In certain aspects, provided is a method of treating an anxiety disorder, an insomnia disorder, a parasomnia, an obsessive-compulsive disorder, an autism spectrum disorder, a disruptive, impulse-control, and conduct (DIC) disorder, an attention-deficit/hyperactivity disorder, a trauma- and stress-related disorder, a psychotic disorder, a bipolar disorder, a depressive disorder, multiple sclerosis, spasticity, epilepsy, Niemann-Pick disease, a substance-related and addictive disorder, a neurocognitive disorder, Tourette's Syndrome, fibromyalgia, or neuropathic pain in a subject in need thereof, comprising administering to the subject any of the compounds and compositions, including the pharmaceutical compositions, as described herein.

It is to be understood that both the Summary and the Detailed Description are exemplary and explanatory only, and are not restrictive of the disclosure as claimed.

### BRIEF DESCRIPTION OF THE FIGURES

The present application can be understood by reference to the following description taken in conjunction with the accompanying figures

FIG. 1 depicts human FAAH activity for exemplary compounds of the disclosure.

FIG. 2 depicts rat FAAH activity for exemplary compounds of the disclosure.

FIG. 3 depicts study schema for exemplary compounds of the disclosure.

FIG. 4 depicts 1H-NMR spectrum for Compound B.

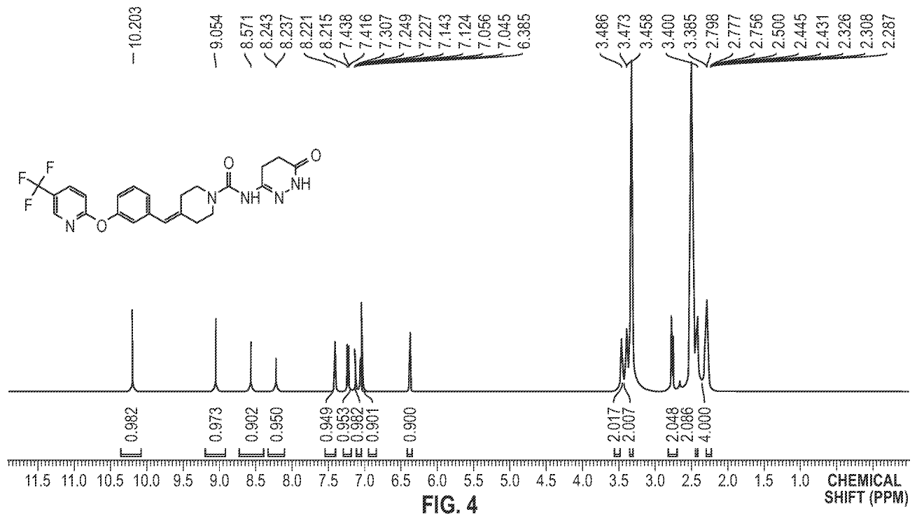

FIG. 5 depicts 1H-NMR spectrum for Compound C.

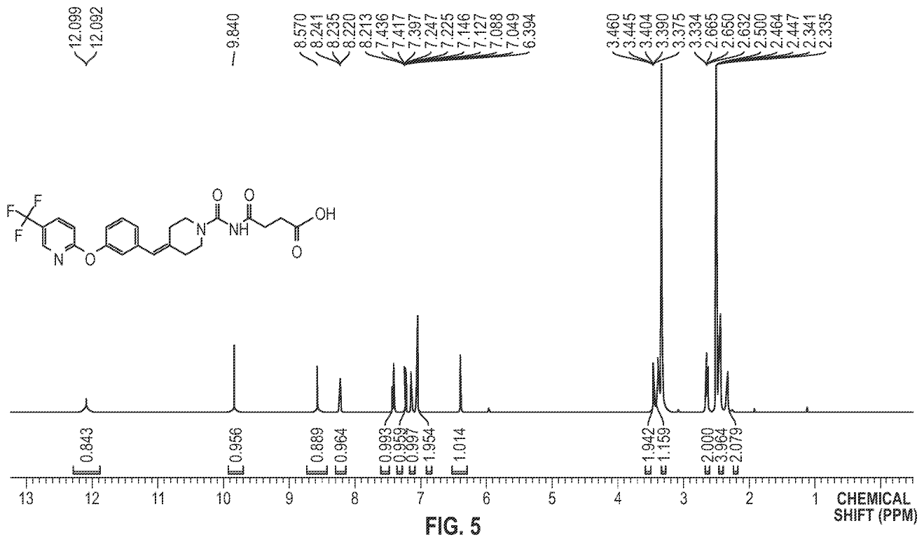

FIG. 6 depicts 1H-NMR spectrum for Compound A-SE.

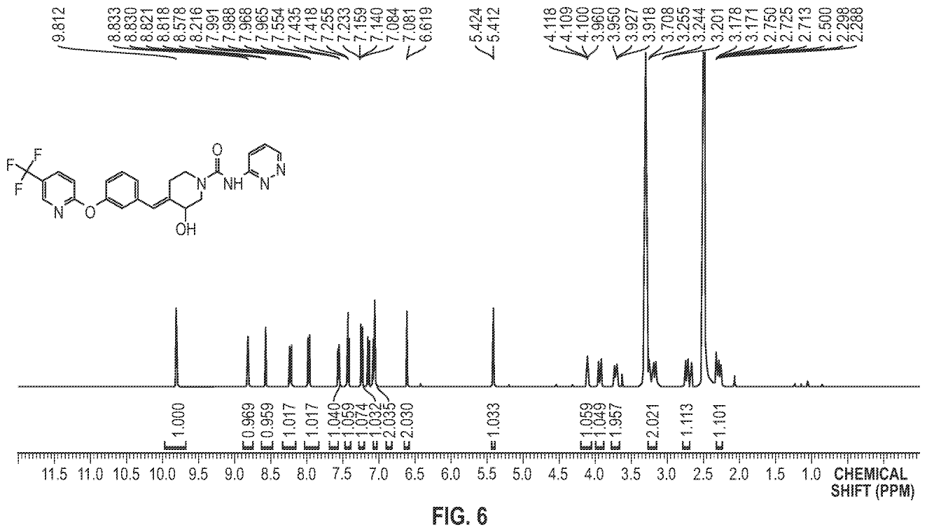

FIG. 7 depicts 1H-NMR spectrum for Compound A-RZ.

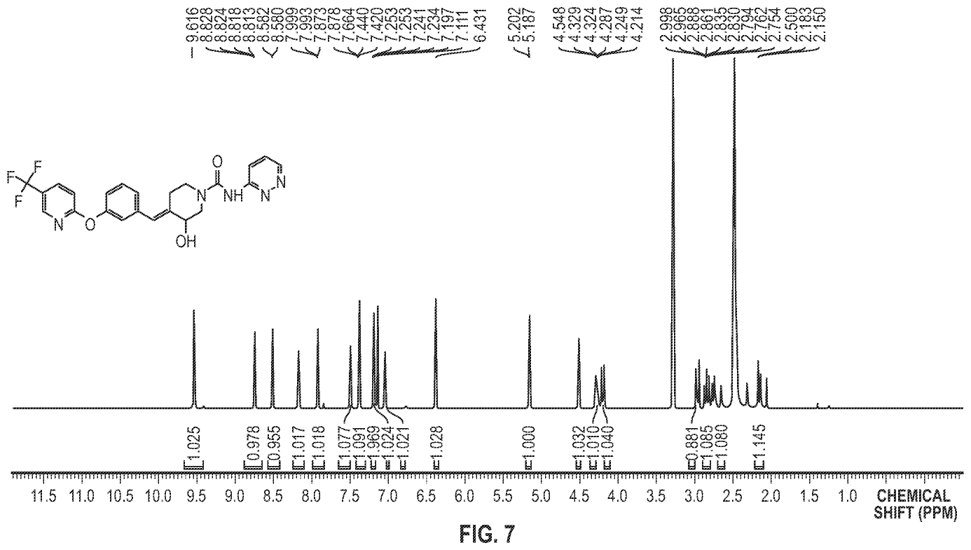

FIG. 8 depicts 1H-NMR spectrum for Compound A-RE.

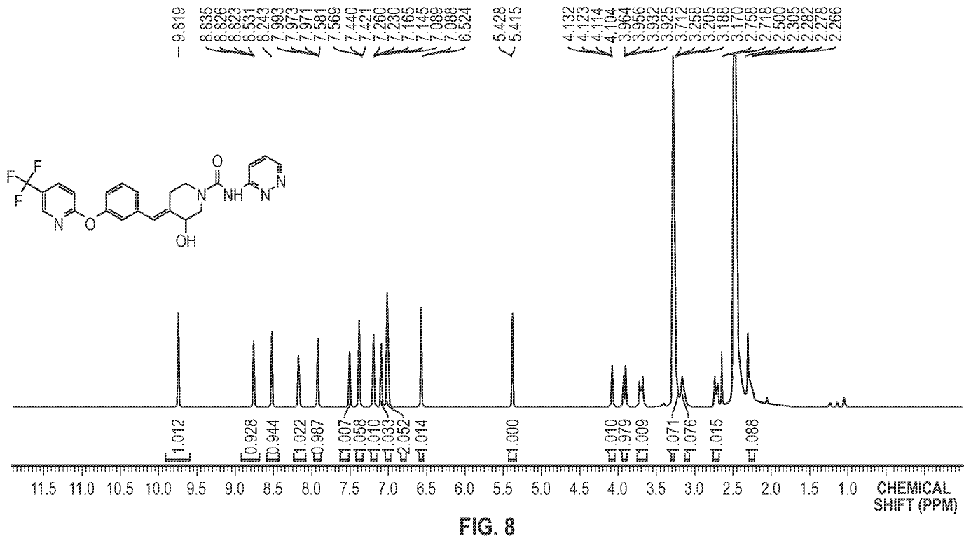

FIG. 9 depicts 1H-NMR spectrum for Compound A-SZ.

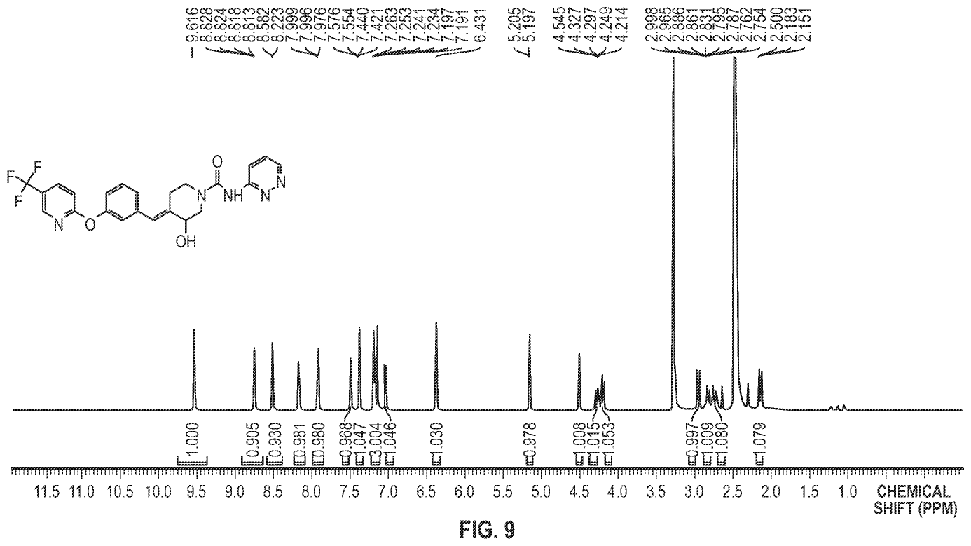

### DETAILED DESCRIPTION

Embodiments of the disclosure relate to pharmaceutical compositions comprising compounds of Formula (I), as well as uses of compounds of Formula (I) and methods of treatment. It has been surprisingly discovered that a compound of Formula (I) can inhibit FAAH.

Various examples and embodiments of the subject matter disclosed are possible and will be apparent to a person of ordinary skill in the art, given the benefit of this disclosure. In this disclosure reference to “some embodiments,” “certain embodiments,” “certain exemplary embodiments” and similar phrases each means that those embodiments are non-limiting examples of the inventive subject matter, and there may be alternative embodiments which are not excluded.

The articles “a,” “an,” and “the” are used herein to refer to one or more than one (i.e., to at least one) of the grammatical object of the article. By way of example, “an element” means one element or more than one element.

As used herein, the term “about” means±10% of the noted value. By way of example only, “about 50 mg” could include from 45 mg to and including 55 mg.

The word “comprising” is used in a manner consistent with its open-ended meaning, that is, to mean that a given product or process can optionally also have additional features or elements beyond those expressly described. It is understood that wherever embodiments are described with the language “comprising,” otherwise analogous embodiments described in terms of “consisting of” and/or “consisting essentially of” are also contemplated and within the scope of this disclosure.

As used herein, the term “alkyl” refers to straight chain or branched chain saturated hydrocarbon groups, generally having a specified number of carbon atoms (e.g., C₁-C₆ alkyl). Examples of alkyl groups include methyl, ethyl, n-propyl, i-propyl, n-butyl, s-butyl, i-butyl, t-butyl, pent-1-yl, pent-2-yl, pent-3-yl, 3-methylbut-1-yl, 3-methylbut-2-yl, 2-methylbut-2-yl, 2,2,2-trimethyleth-1-yl, n-hexyl, and the like.

As used herein, the term “aryl” refers to o monocyclic or bicyclic monovalent and divalent aromatic carbocyclic groups, such as phenyl, biphenyl or naphthyl groups.

As used herein, the term “cycloalkyl” refers to saturated monocyclic and bicyclic hydrocarbon rings, generally having a specified number of carbon atoms that comprise the ring (e.g., C₃-C₇ cycloalkyl). Monocyclic cycloalkyl groups can be cyclopropyl, cyclobutyl, cyclopentyl, cyclohexyl, and the like. Bicyclic cycloalkyl groups can be bicyclo[1.1.0]butyl, bicyclo[1.1.1]pentyl, bicyclo[2.1.0]pentyl, bicyclo[2.1.1]hexyl, bicyclo[3.1.0]hexyl, bicyclo[2.2.1]heptyl, bicyclo[3.2.0]heptyl, bicyclo[3.1.1]heptyl, bicyclo[4.1.0]heptyl, bicyclo[2.2.2]octyl, bicyclo[3.2.1]octyl, bicyclo[4.1.1]octyl, bicyclo[3.3.0]octyl, bicyclo[4.2.0]octyl, bicyclo[3.3.1]nonyl, bicyclo[4.2.1]nonyl, bicyclo[4.3.0]nonyl, bicyclo[3.3.2]decyl, bicyclo[4.2.2]decyl, bicyclo[4.3.1]decyl, bicyclo[4.4.0]decyl, bicyclo[3.3.3]undecyl, bicyclo[4.3.2]undecyl, bicyclo[4.3.3]dodecyl, and the like.

As used herein, the term “halo” or “halogen” refer to fluoro, chloro, bromo, and iodo.

As used herein, the term “heteroaryl” refers to monovalent or divalent aromatic groups, respectively, containing from 1 to 4 ring heteroatoms selected from O, S, or N. Monocyclic (and monovalent) aryl groups can be pyrrolyl, furanyl, thiopheneyl, pyrazolyl, imidazolyl, isoxazolyl, oxazolyl, isothiazolyl, thiazolyl, 1,2,3-triazolyl, 1,3,4-triazolyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, tetrazolyl, pyridinyl, pyridazinyl, pyrimidinyl, pyrazinyl, and the like. Heteroaryls can be bicyclic groups, tricyclic groups, including fused ring systems wherein at least one ring is aromatic. Multicyclic (and monovalent) aryl groups can be pyrenyl, carbazolyl, benzofuranyl, benzothiopheneyl, indolyl, benzoxazolyl, benzodioxazolyl, benzimidazolyl, indazolyl, benzotriazolyl, benzothiofuranyl, benzothiazolyl, benzotriazolyl, benzotetrazolyl, benzoisoxazolyl, benzoisothiazolyl, benzoimidazolinyl, pyrrolo[2,3-b]pyridinyl, pyrrolo[2,3-c]pyridinyl, pyrrolo[3,2-c]pyridinyl, pyrrolo[3,2-b]pyridinyl, imidazo[4,5-b]pyridinyl, imidazo[4,5-c]pyridinyl, pyrazolo[4,3-c]pyridinyl, pyrazolo[4,3-c]pyridinyl, pyrazolo[3,4-c]pyridinyl, pyrazolo[3,4-b]pyridinyl, isoindolyl, indazolyl, purinyl, indolizinyl, imidazo[1,2-a]pyridinyl, imidazo[1,5-a]pyridinyl, pyrazolo[1,5-a]pyridinyl pyrrolo[1,2-b]pyridinyl, and imidazo[1,2-c]pyridinyl. Other heteroaryls can be quinolinyl, isoquinolinyl, cinnolinyl, quinazolinyl, quinoxalinyl, phthalazinyl, 1,6-naphthyridinyl, 1,7-naphthyridinyl, 1,8-naphthyridinyl, 1,5-naphthyridinyl, 2,6-naphthyridinyl, 2,7-naphthyridinyl, pyrido[3,2-d]pyrimidinyl, pyrido[4,3-d]pyrimidinyl, pyrido[3,4-d]pyrimidinyl, pyrido[2,3-d]pyrimidinyl, pyrido[2,3-b]pyrazinyl, pyrido[3,4-b]pyrazinyl, pyrimido[5,4-d]pyrimidinyl, pyrazino[2,3-b]pyrazinyl, pyrimido[4,5-c]pyrimidinyl, phenanthridinyl, phenanthrolinyl, phenazinyl, phenothiazinyl, phenoxathiinyl, phenoxazinyl, acridinyl, azocinyl, 4aH-carbazolyl, chromanyl, chromenyl, indolenyl, indolinyl, 3H-indolyl, isobenzofuranyl, isochromanyl, isoindazolyl, isoindolinyl, pyrimidinyl, pteridinyl, phthalazinyl, purinyl, pyridazinyl, pyrazinyl, pyridooxazole, pyridoimidazole, pyridothiazole, pyridyl, pyridopyrimidinyl, quinoxalinyl, quinazolinyl, thianthrenyl, xanthenyl, and the like.

As used herein, the terms “excipient” or “adjuvant” refer to any substance in a pharmaceutical formulation that is not an active pharmaceutical ingredient (API).

As used herein, the term “pharmaceutical composition” refers to the combination of one or more drug substances and one or more excipients.

As used herein, the terms “drug product,” “pharmaceutical dosage form,” “dosage form,” “final dosage form,” and the like refer to a pharmaceutical composition that can be administered to a subject in need of the treatment and can be in the form of tablets, capsules, liquid solutions or suspensions, patches, films, and the like.

As used herein, the term “subject” refers to a mammal, including humans.

As used herein, the term “therapeutically effective amount” refers to the quantity of a compound that can be useful for treating a subject. The amount can depend on a variety of factors, including the weight and age of the subject and the route of administration, among other factors.

As used herein, the term “treating” refers to reversing, alleviating, inhibiting the progress of, or preventing a disorder or condition to which such term applies, or to reversing, alleviating, inhibiting the progress of, or preventing one or more symptoms of such disorder or condition.

### Compounds of the Disclosure

In certain aspects, embodiments of the disclosure relate to a compound of Formula (I):

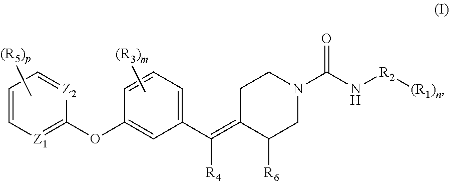

or a pharmaceutically acceptable salt or solvate thereof,
wherein:
each R₁ is independently hydrogen, —C₁-C₆ alkyl, —OH, or —O—(C₁-C₆ alkyl);
R₂ is —C(O)—C₁-C₆ alkyl-C(O)—R₇, a 6-membered aromatic heterocycle containing 1 or 2 nitrogen ring heteroatoms, or dihydropyridazinone, wherein R₇ is hydrogen, halogen, —C₁-C₆ alkyl, —OH, —O—C₁-C₆ alkyl, or —O-haloalkyl;
R₆ is hydrogen or —OH;
each R₃ is independently hydrogen, halogen, —C₁-C₆ alkyl, —(CH₂)₀₋₃—C₃-C₆ cycloalkyl, or —O—C₁-C₆ alkyl;
R₄ is hydrogen, —OH, —C₁-C₆ alkyl, phenyl, or halogen;
each R₅ is independently hydrogen, halogen, haloalkyl, —O-haloalkyl, —C₁-C₆ alkyl, —C(O)C₁-C₆ alkyl, —O—C₁-C₆ alkyl, —S—C₁-C₆ alkyl, —(CH₂)₀₋₃—C₃-C₆ cycloalkyl, CN, aryl, and heteroaryl; wherein the —C₁-C₆ alkyl, —O(C₁-C₆ alkyl), —(CH₂)₀₋₃—C₃-C₆ cycloalkyl, aryl, and heteroaryl groups being optionally independently substituted with from 1 to 4 —C₁-C₆ alkyl, —OH, or halogen substituents;
m is 0, 1, 2, 3, or 4;
n is 0, 1, 2, 3, or 4;
p is 0, 1, 2, 3, or 4; and
Z₁ and Z₂ are independently selected from N and CH,
with the proviso that when R₂ is a 6-membered aromatic heterocycle containing 1 or 2 nitrogen ring heteroatoms, R₆ is not H.

In certain embodiments, Z₁ can be N and Z₂ can be CH.

In certain embodiments, R₅ can be CF₃.

In certain embodiments, Z₁ can be N, Z₂ can be CH, p can be 1, and R₅ can be CF₃.

In certain embodiments, R₆ can be —OH.

In certain embodiments, R₂ can be pyridazine.

In certain embodiments, R₆ can be H and R₂ can be —C(O)—C₁-C₆ alkyl-C(O)—R₇ or dihydropyridazinone.

In certain embodiments, n can be 0.

In certain embodiments, a compound of Formula (I) is a compound of Formula (I-A):

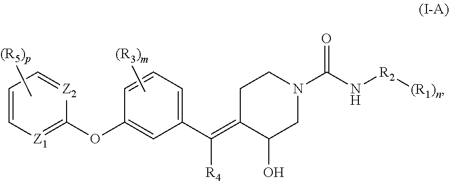

or a pharmaceutically acceptable salt or solvate thereof, wherein R₁, R₂, R₃, R₄, R₅, Z₁, Z₂, n, m, and p are as defined in Formula (I). In some embodiments, in conjunction with embodiments above or below, n is 0 and R₂ is 6-membered aromatic heterocycle containing 1 or 2 nitrogen ring heteroatoms. In some embodiments, in conjunction with embodiments above or below, Z₁ is N and Z₂ is CH. In some embodiments, in conjunction with embodiments above or below, p is 1 and R₅ is CF₃.

In certain embodiments, a compound of Formula (I) is a compound of Formula (I-B):

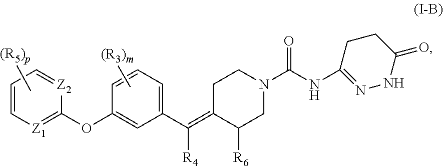

or a pharmaceutically acceptable salt or solvate thereof, wherein R₃, R₄, R₅, R₆, Z₁, Z₂, n, m, and p are as defined in Formula (I). In some embodiments, in conjunction with embodiments above or below, Z₁ is N and Z₂ is CH. In some embodiments, in conjunction with embodiments above or below, p is 1 and R₅ is CF₃.

In certain embodiments, a compound of Formula (I) is a compound of Formula (I-C):

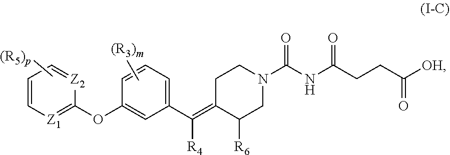

or a pharmaceutically acceptable salt or solvate thereof, wherein R₃, R₄, R₅, R₆, Z₁, Z₂, n, m, and p are as defined in Formula (I). In some embodiments, in conjunction with embodiments above or below, Z₁ is N and Z₂ is CH. In some embodiments, in conjunction with embodiments above or below, p is 1 and R₅ is CF₃.

In certain embodiments, the compound of Formula (I) is

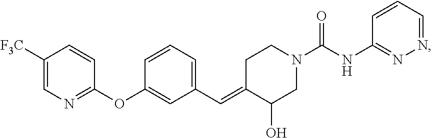 [SMILES: O=C(Nc1cccnn1)N1CC/C(=C\c2cccc(Oc3ccc(C(F)(F)F)cn3)c2)C(O)C1]

also referred to as 3-hydroxy-N-(pyridazin-3-yl)-4-(3-((5-trifluoromethyl)pyridine-2-yl)oxy)benzylidene)piperidine-1-carboxamide or “Compound A”, or a pharmaceutically acceptable salt or solvate thereof.

In certain embodiments, wherein the compound of Formula (I) is:
(R,E)-3-hydroxy-N-(pyridazin-3-yl)-4-(3-((5-(trifluoromethyl)pyridin-2-yl)oxy)benzylidene)piperidine-1-carboxamide (also referred to herein as “Compound A-RE”);
(S,E)-3-hydroxy-N-(pyridazin-3-yl)-4-(3-((5-(trifluoromethyl)pyridin-2-yl)oxy)benzylidene)piperidine-1-carboxamide (also referred to herein as “Compound A-SE”);
(R,Z)-3-hydroxy-N-(pyridazin-3-yl)-4-(3-((5-(trifluoromethyl)pyridin-2-yl)oxy)benzylidene)piperidine-1-carboxamide (also referred to herein as “Compound A-RZ”); or
(S,Z)-3-hydroxy-N-(pyridazin-3-yl)-4-(3-((5-(trifluoromethyl)pyridin-2-yl)oxy)benzylidene)piperidine-1-carboxamide (also referred to herein as “Compound A-SZ”), or a pharmaceutically acceptable salt or solvate thereof.

In certain embodiments, wherein the compound of Formula (I) is

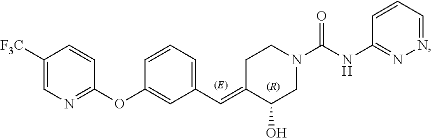

(R,E)-3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof.

In certain embodiments, wherein the compound of Formula (I) can be

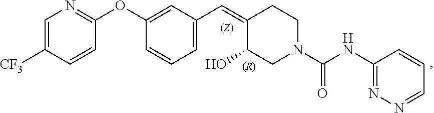

also referred to as (R,Z)-3-hydroxy-N-(pyridazin-3-yl)-4-(3-((5-(trifluoromethyl)pyri din-2-yl)oxy)benzylidene)piperidine-1-carboxamide, or a pharmaceutically acceptable salt or solvate thereof.

In certain embodiments, the compound of Formula (I) is

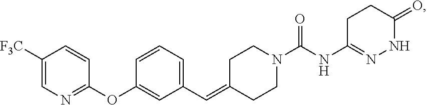 [SMILES: O=C1CCC(NC(=O)N2CCC(=Cc3cccc(Oc4ccc(C(F)(F)F)cn4)c3)CC2)=NN1]

also referred to as N-(6-oxo-1,4,5,6-tetrahydropyridazin-3-yl)-4-(3-((5-(trifluoromethyl)pyridin-2-yl)oxy)benzylidene)piperidine-1-carboxamide or “Compound B”, or a pharmaceutically acceptable salt or solvate thereof.

In certain embodiments, the compound of Formula (I) is

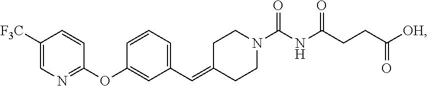 [SMILES: O=C(O)CCC(=O)NC(=O)N1CCC(=Cc2cccc(Oc3ccc(C(F)(F)F)cn3)c2)CC1]

also referred to as 4-oxo-4-(4-(3-((5-(trifluoromethyl)pyridin-2-yl)oxy)benzylidene)piperidine-1-carboxamido)butanoic acid or “Compound C”, or a pharmaceutically acceptable salt or solvate thereof.

In some variations, compounds of Formula (I), including the compounds specifically named above, can form pharmaceutically acceptable complexes, salts, solvates and hydrates. The salts can acid addition salts (including di-acids) and base salts.

Pharmaceutically acceptable acid addition salts can be salts derived from inorganic acids such as hydrochloric acid, nitric acid, phosphoric acid, sulfuric acid, hydrobromic acid, hydroiodic acid, hydrofluoric acid, and phosphorous acids, as well salts derived from organic acids, such as aliphatic mono- and dicarboxylic acids, phenyl-substituted alkanoic acids, hydroxy alkanoic acids, alkanedioic acids, aromatic acids, aliphatic, and aromatic sulfonic acids, etc. Such salts can be acetate, adipate, aspartate, benzoate, besylate, bicarbonate, carbonate, bisulfate, sulfate, borate, camsylate, citrate, cyclamate, edisylate, esylate, formate, fumarate, gluceptate, gluconate, glucuronate, hexafluorophosphate, hibenzate, hydrochloride, chloride, hydrobromide, bromide, hydroiodide, iodide, isethionate, lactate, malate, maleate, malonate, mesylate, methylsulfate, naphthylate, 2-napsylate, nicotinate, nitrate, orotate, oxalate, almitate, pamoate, phosphate, hydrogen phosphate, dihydrogen phosphate, pyroglutamate, saccharate, stearate, succinate, tannate, tartrate, tosylate, trifluoroacetate, and xinofoate salts.

Pharmaceutically acceptable base salts include salts derived from bases, including metal cations, such as an alkali or alkaline earth metal cation, as well as amines. Examples of suitable metal cations include sodium (Na⁺), potassium (K⁺), magnesium (Mg²⁺), calcium (Ca²⁺), zinc (Zn²⁺), and aluminum (Al³⁺). Suitable amines can be arginine, N,N′-dibenzylethylenediamine, chloroprocaine, choline, diethylamine, diethanolamine, dicyclohexyla mine, ethylenediamine, glycine, lysine, N-methylglucamine, olamine, 2-amino-2-hydroxymethyl-propane-1,3-diol, and procaine. For a discussion of useful acid addition and base salts, see S. M. Berge et al., “Pharmaceutical Salts,” 66 J. Pharm. Sci, 1-19 (1977); see also Stahl and Wermuth, Handbook of Pharmaceutical Salts: Properties, Selection, and Use (2002), which are herein incorporated by reference in their entirety.

Pharmaceutically acceptable salts can be prepared using various methods. For example, one can react a compound with an appropriate acid or base to give the desired salt. One can also react a precursor of the compound with an acid or base to remove an acid- or base-labile protecting group or to open a lactone or lactam group of the precursor. Additionally, one can convert a salt of the compound to another salt through treatment with an appropriate acid or base or through contact with an ion exchange resin. Following reaction, one can then isolate the salt by filtration if it precipitates from solution, or by evaporation to recover the salt. The degree of ionization of the salt can vary from completely ionized to almost non-ionized.

The compounds herein, and the pharmaceutically acceptable salts thereof, can exist in a continuum of solid states ranging from fully amorphous to fully crystalline. They can also exist in unsolvated and solvated forms. The term “solvate” describes a molecular complex comprising the compound and one or more pharmaceutically acceptable solvent molecules (e.g., ethanol). The term “hydrate” is a solvate in which the solvent is water. Pharmaceutically acceptable solvates can be those in which the solvent can be isotopically substituted (e.g., D₂O, d₆-acetone, d₆-DMSO).

A classification system for solvates and hydrates of organic compounds can be one that distinguishes between isolated site, channel, and metal-ion coordinated solvates and hydrates. See, e.g., K. R. Morris (H. G. Brittain ed.) Polymorphism in Pharmaceutical Solids (1995), which is herein incorporated by reference in its entirety. Isolated site solvates and hydrates can be ones in which the solvent (e.g., water) molecules are isolated from direct contact with each other by intervening molecules of the organic compound. In channel solvates, the solvent molecules can lie in lattice channels where they are next to other solvent molecules. In metal-ion coordinated solvates, the solvent molecules can be bonded to the metal ion.

When the solvent or water is tightly bound, the complex can have a well-defined stoichiometry independent of humidity. When, however, the solvent or water is weakly bound, as in channel solvates and in hygroscopic compounds, the water or solvent content can depend on humidity and drying conditions. In such cases, non-stoichiometry can be the norm.

The compounds herein, and the pharmaceutically acceptable salts thereof, can also exist as multi-component complexes (other than salts and solvates) in which the compound and at least one other component can be present in stoichiometric or non-stoichiometric amounts. Complexes of this type can be clathrates (drug-host inclusion complexes) and co-crystals. The latter can be typically defined as crystalline complexes of neutral molecular constituents which can be bound together through non-covalent interactions, but could also be a complex of a neutral molecule with a salt. Co-crystals can be prepared by melt crystallization, by recrystallization from solvents, or by physically grinding the components together. See, e.g., 0. Almarsson and M. J. Zaworotko, Chem. Commun., 17:1889-1896 (2004), which is herein incorporated by reference in its entirety. For a general review of multi-component complexes, see J. K. Haleblian, J. Pharm. Sci. 64(8):1269-88 (1975), which is herein incorporated by reference in its entirety.

Geometrical (cis/trans) isomers can be separated by techniques such as chromatography and fractional crystallization.

“Tautomers” refer to structural isomers that can be interconvertible via a low energy barrier. Tautomeric isomerism (tautomerism) can take the form of proton tautomerism in which the compound can contain, for example, an imino, keto, or oxime group, or valence tautomerism in which the compound can contain an aromatic moiety.

Compounds described herein also include all pharmaceutically acceptable isotopic variations, in which at least one atom is replaced by an atom having the same atomic number, but an atomic mass different from the atomic mass usually found in nature. Isotopes suitable for inclusion in the compounds herein, and the pharmaceutically acceptable salts thereof can be, for example, isotopes of hydrogen, such as ²H and ³H; isotopes of carbon, such as ¹¹C, ¹³C and ¹⁴C; isotopes of nitrogen, such as ¹³N and ¹⁵N; isotopes of oxygen, such as ¹⁵O, ¹⁷O and ¹⁸O; isotopes of sulfur, such as ³⁵S; isotopes of fluorine, such as ¹⁸F; isotopes of chlorine, such as ³⁶Cl, and isotopes of iodine, such as ¹²³I and ¹²⁵I. Use of isotopic variations (e.g., deuterium, ²H) can afford certain therapeutic advantages resulting from greater metabolic stability, for example, increased in vivo half-life or reduced dosage requirements. Additionally, certain isotopic variations of the disclosed compounds can incorporate a radioactive isotope (e.g., tritium, ³H, or ¹⁴C), which can be useful in drug and/or substrate tissue distribution studies. Substitution with positron emitting isotopes, such as ¹¹C, ¹⁸F, ¹⁵O and ¹³N, can be useful in Positron Emission Topography (PET) studies for examining substrate receptor occupancy. Isotopically-labeled compounds can be prepared by processes analogous to those described elsewhere in the disclosure using an appropriate isotopically-labeled reagent in place of a non-labeled reagent.

### Compositions

In certain aspects, provided is a composition comprising a compound of Formula (I), including any of the compounds specifically named above, or a pharmaceutically acceptable salt or solvate thereof, wherein at least 70%, at least 80%, at least 85%, at least 90%, at least 95%, or at least 99% by weight of the composition is the compound, or a pharmaceutically acceptable salt or solvate thereof. In certain embodiments, the composition has less than 30%, less than 20%, less than 15%, less than 10%, less than 5%, or less than 1% by weight of impurities and/or by products from the synthesis of the compound, or a pharmaceutically acceptable salt or solvate thereof. Any suitable techniques and methods known in the art may be employed to determine purity of the composition, including nuclear magnetic resonance, mass spectrometry, etc.

In some embodiments, the composition comprises a compound of Formula (I-A), or a pharmaceutically acceptable salt or solvate thereof. In some embodiments, the composition comprises Compound A. In some embodiments, the composition comprises Compound A-RE. In some embodiments, the composition comprises Compound A-SE. In some embodiments, the composition comprises Compound A-RZ. In some embodiments, the composition comprises Compound A-SZ. In some embodiments, the composition comprises a compound of Formula (I-B), or a pharmaceutically acceptable salt or solvate thereof. In some embodiments, the composition comprises compound B. In some embodiments, the composition comprises a compound of Formula (I-C), or a pharmaceutically acceptable salt or solvate thereof. In some embodiments, the composition comprises compound C.

### Pharmaceutical Compositions

In certain aspects, provided is a pharmaceutical composition comprising a compound of Formula (I), including any of the compounds specifically named above; and at least one pharmaceutically acceptable excipient. In some variations, the pharmaceutical composition is a solid oral dosage form. In one variation, the pharmaceutical composition is a tablet.

In some embodiments, the pharmaceutical composition comprises a compound of Formula (I-A), or a pharmaceutically acceptable salt or solvate thereof, and at least one pharmaceutically acceptable excipient. In some embodiments, the pharmaceutical composition comprises Compound A. In some embodiments, the pharmaceutical composition comprises Compound A-RE. In some embodiments, the pharmaceutical composition comprises Compound A-SE. In some embodiments, the pharmaceutical composition comprises Compound A-RZ. In some embodiments, the pharmaceutical composition comprises Compound A-SZ. In some embodiments, the pharmaceutical composition comprises a compound of Formula (I-B), or a pharmaceutically acceptable salt or solvate thereof, and at least one pharmaceutically acceptable excipient. In some embodiments, the pharmaceutical composition comprises compound B. In some embodiments, the pharmaceutical composition comprises a compound of Formula (I-C), or a pharmaceutically acceptable salt or solvate thereof, and at least one pharmaceutically acceptable excipient. In some embodiments, the pharmaceutical composition comprises compound C.

In certain embodiments, the pharmaceutical composition comprises 3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof; and at least one pharmaceutically acceptable excipient.

In certain embodiments, the pharmaceutical composition comprises (R,E)-3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof; and at least one pharmaceutically acceptable excipient.

In certain embodiments, the pharmaceutical composition comprises (S,E)-3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof; and at least one pharmaceutically acceptable excipient.

In certain embodiments, the pharmaceutical composition comprises (R,Z)-3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof; and at least one pharmaceutically acceptable excipient.

In certain embodiments, the pharmaceutical composition comprises (S,Z)-3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof and at least one pharmaceutically acceptable excipient.

In certain embodiments, the pharmaceutical composition comprises N-(6-oxo-1,4,5,6-tetrahydropyridazin-3-yl)-4-(3-((5-(trifluoromethyl)pyridin-2-yl)oxy)benzylidene)piperidine-1-carboxamide, or a pharmaceutically acceptable salt or solvate thereof and at least one pharmaceutically acceptable excipient.

In certain embodiments, the pharmaceutical composition comprises 4-oxo-4-(4-(3-((5-(trifluoromethyl)pyridin-2-yl)oxy)benzylidene)piperidine-1-carboxamido)butanoic acid, or a pharmaceutically acceptable salt or solvate thereof and at least one pharmaceutically acceptable excipient.

In certain embodiments, the pharmaceutical composition comprises N-(pyridazin-3-yl)-4-(3-((5-(trifluoromethyl)pyridin-2-yl)oxy)benzylidene)piperidine-1-carboxamide, or a pharmaceutically acceptable salt or solvate thereof and at least one pharmaceutically acceptable excipient.

The compounds herein, and the pharmaceutically acceptable salts thereof, can be administered as crystalline or amorphous forms, hydrates, solvates, complexes, and tautomers thereof, as well as all isotopically-labeled compounds thereof. They can be administered alone or in combination with one another or with one or more pharmacologically active compounds which are different than the compounds described or specifically named herein, and the pharmaceutically acceptable salts thereof. One or more these compounds can be administered as a pharmaceutical composition (a formulation) in association with one or more pharmaceutically acceptable excipients. The choice of excipients depends on the particular mode of administration, the effect of the excipient on solubility and stability, and the nature of the dosage form, among other things. Useful pharmaceutical compositions and methods for their preparation can be found, for example, in A. R. Gennaro (ed.), Remington: The Science and Practice of Pharmacy (20th ed., 2000), which is herein incorporated by reference in its entirety.

The compounds herein, and the pharmaceutically acceptable salts thereof, can be administered orally. Oral administration can involve swallowing in which case the compound enters the bloodstream via the gastrointestinal tract. Alternatively or additionally, oral administration can involve mucosal administration (e.g., buccal, sublingual, supralingual administration) such that the compound enters the bloodstream through the oral mucosa.

Formulations suitable for oral administration can be solid, semi-solid, and liquid systems, such as tablets; soft or hard capsules containing multi- or nano-particulates, liquids, or powders; lozenges which can be liquid-filled; chews; gels; fast dispersing dosage forms; films; ovules; sprays; and buccal or mucoadhesive patches.

Liquid formulations can be suspensions, solutions, syrups, and elixirs. These formulations can be employed as fillers in soft or hard capsules (made, for example, from gelatin or hydroxypropyl methylcellulose) and can comprise a carrier (e.g., water, ethanol, polyethylene glycol, propylene glycol, methylcellulose, or a suitable oil) and one or more emulsifying agents, suspending agents or both. Liquid formulations can also be prepared by the reconstitution of a solid (e.g., from a sachet).

The compounds herein, and the pharmaceutically acceptable salts thereof, may also be used in fast-dissolving, fast-disintegrating dosage forms such as those described in Liang and Chen, Expert Opinion in Therapeutic Patents, 11 (6):981-986 (2001), which is herein incorporated by reference in its entirety.

For tablet dosage forms, depending on dose, the active pharmaceutical ingredient (API) can comprise from about 1 wt % to about 80 wt % of the dosage form and can comprise from about 5 wt % to about 60 wt % of the dosage form. In addition to the API, tablets can include one or more disintegrants, binders, diluents, surfactants, glidants, lubricants, anti oxidants, colorants, flavoring agents, preservatives, and taste-masking agents. Examples of disintegrants include sodium starch glycolate, sodium carboxymethyl cellulose, calcium carboxymethyl cellulose, croscarmellose sodium, crospovidone, polyvinylpyrrolidone, methyl cellulose, microcrystalline cellulose, C₁-C₆ alkyl-substituted hydroxypropylcellulose, starch, pregelatinized starch, and sodium alginate. The disintegrant can comprise from about 1 wt % to about 25 wt % or from about 5 wt % to about 20 wt % of the dosage form. Binders can be used to impart cohesive qualities to a tablet formulation. Suitable binders include microcrystalline cellulose, gelatin, sugars, polyethylene glycol, natural and synthetic gums, polyvinylpyrrolidone, pregelatinized starch, hydroxypropylcellulose, and hydroxypropylmethylcellulose. Tablets can also contain diluents, such as lactose (monohydrate, spray-dried monohydrate, anhydrous), mannitol, xylitol, dextrose, sucrose, sorbitol, microcrystalline cellulose, starch, and dibasic calcium phosphate dihydrate

Tablets can comprise surface active agents, such as sodium lauryl sulfate and polysorbate 80, and glidants such as silicon dioxide and talc. When present, surface active agents can comprise from about 0.2 wt % to about 5 wt % of the tablet, and glidants can comprise from about 0.2 wt % to about 1 wt % of the tablet. Tablets can contain lubricants such as magnesium stearate, calcium stearate, zinc stearate, sodium stearyl fumarate, and mixtures of magnesium stearate with sodium lauryl sulfate.

Lubricants can comprise from about 0.25 wt % to about 10 wt % or from about 0.5 wt % to about 3 wt % of the tablet.

Tablet blends can be compressed directly or by roller compaction to form tablets. Tablet blends or portions of blends can alternatively be wet-, dry-, or melt-granulated, melt congealed, or extruded before tableting. If desired, prior to blending one or more of the components can be sized by screening or milling or both. The final dosage form can comprise one or more layers and can be coated, uncoated, or encapsulated. Exemplary tablets can contain up to about 80 wt % of API, from about 10 wt % to about 90 wt % of binder, from about 0 wt % to about 85 wt % of diluent, from about 2 wt % to about 10 wt % of disintegrant, and from about 0.25 wt % to about 10 wt % of lubricant. For a discussion of blending, granulation, milling, screening, tableting, coating, as well as a description of techniques for preparing drug products, see A. R. Gennaro (ed.), Remington: The Science and Practice of Pharmacy (20th ed., 2000); H. A. Lieberman et al. (ed.), Pharmaceutical Dosage Forms: Tablets, Vol. 1-3 (2d ed., 1990); and D. K. Parikh & C. K. Parikh, Handbook of Pharmaceutical Granulation Technology, Vol. 81 (1997), which are herein incorporated by reference in their entirety.

Consumable oral films for human or veterinary use can be pliable water-soluble or water-swellable thin film dosage forms that can be rapidly dissolving or mucoadhesive. In addition to the API, a typical film can contain one or more film-forming polymers, binders, solvents, humectants, plasticizers, stabilizers or emulsifiers, viscosity-modifying agents, and solvents. Other film ingredients can be anti-oxidants, colorants, flavorants and flavor enhancers, preservatives, salivary stimulating agents, cooling agents, co-solvents (including oils), emollients, bulking agents, anti-foaming agents, surfactants, and taste-masking agents. Some components of the formulation can perform more than one function.

In addition to dosing, the amount of API in the film can depend on its solubility. If water soluble, the API can comprise from about 1 wt % to about 80 wt % of the non-solvent components (solutes) in the film or from about 20 wt % to about 50 wt % of the solutes in the film. A less soluble API can comprise a greater proportion of the composition, such as up to about 88 wt % of the non-solvent components in the film. The film-forming polymer can be natural polysaccharides, proteins, or synthetic hydrocolloids and can comprise from about 0.01 wt % to about 99 wt % or from about 30 wt % to about 80 wt % of the film. Film dosage forms can be prepared by evaporative drying of thin aqueous films coated onto a peelable backing support or paper, which can carried out in a drying oven or tunnel (e.g., in a combined coating-drying apparatus), in lyophilization equipment, or in a vacuum oven.

Solid formulations for oral administration can include immediate release formulations and modified release formulations. Modified release formulations can include delayed-, sustained-, pulsed-, controlled-, targeted-, and programmed-release. For a general description of suitable modified release formulations, see U.S. Pat. No. 6,106,864. For details of other useful release technologies, such as high energy dispersions and osmotic and coated particles, see Verma et al, Pharmaceutical Technology On-line (2001) 25(2):1-14, which is herein incorporated by reference in its entirety. Compounds of Formula (I), including the compounds specifically named above, and the pharmaceutically acceptable salts thereof can also be administered directly into the blood stream, muscle, or an internal organ of the subject. Suitable techniques for parenteral administration can include intravenous, intraarterial, intraperitoneal, intrathecal, intraventricular, intraurethral, intrasternal, intracranial, intramuscular, intrasynovial, and subcutaneous administration. Suitable devices for parenteral administration can include needle injectors, including microneedle injectors, needle-free injectors, and infusion devices.

Parenteral formulations can be aqueous solutions that can contain excipients, such as salts, carbohydrates, and buffering agents (e.g., pH of from about 3 to about 9). For some applications, compounds of Formula (I), including compounds specifically named above, and the pharmaceutically acceptable salts thereof can be formulated as a sterile non-aqueous solution or as a dried form to be used in conjunction with a suitable vehicle, such as sterile, pyrogen-free water. Preparation of parenteral formulations can be under sterile conditions (e.g., by lyophilization).

The solubility of compounds used in the preparation of parenteral solutions can be increased through appropriate formulation techniques, such as the incorporation of solubility-enhancing agents. Formulations for parenteral administration can be formulated to be immediate or modified release. Modified release formulations can include delayed, sustained, pulsed, controlled, targeted, and programmed release. Thus, compounds of Formula (I), including the compounds specifically named above, and the pharmaceutically acceptable salts thereof can be formulated as a suspension, a solid, a semi-solid, or a thixotropic liquid for administration as an implanted depot providing modified release of the active compound. Examples of such formulations can include drug-coated stents and semi-solids and suspensions comprising drug-loaded poly(DL-lactic-coglycolic)acid (PGLA) microspheres.

The compounds herein, and the pharmaceutically acceptable salts thereof, can also be administered topically, intradermally, or transdermally to the skin or mucosa. Formulations for this purpose can include gels, hydrogels, lotions, solutions, creams, ointments, dusting powders, dressings, foams, films, skin patches, wafers, implants, sponges, fibers, bandages, and microemulsions. Liposomes can also be used. Carriers can include alcohol, water, mineral oil, liquid petrolatum, white petrolatum, glycerin, polyethylene glycol, and propylene glycol. Topical formulations can also include penetration enhancers. See, e.g., Finnin and Morgan, J. Pharm. Sci. 88(10):955-958 (1999), which is herein incorporated by reference in its entirety. Topical administration can include delivery by electroporation, iontophoresis, phonophoresis, sonophoresis, and microneedle or needle-free injection. Formulations for topical administration can be formulated to be immediate or modified release as described above.

The compounds herein, and the pharmaceutically acceptable salts thereof, can also be administered intranasally or by inhalation, such as in the form of a dry powder, an aerosol spray, or nasal drops. An inhaler can be used to administer the dry powder, which comprises the API alone, a powder blend of the API and a diluent, such as lactose, or a mixed component particle that includes the API and a phospholipid, such as phosphatidylcholine. For intranasal use, the powder can include a bioadhesive agent, e.g., chitosan or cyclodextrin. A pressurized container, pump, sprayer, atomizer, or nebulizer, can be used to generate an aerosol spray from a solution or suspension comprising the API, one or more agents for dispersing, solubilizing, or extending the release of the API (e.g., EtOH with or without water), one or more solvents (e.g., 1,1,1,2-tetrafluoroethane or 1,1,1,2,3,3,3-heptafluoropropane) that can be a propellant, and an optional surfactant, such as sorbitan trioleate, oleic acid, or an oligolactic acid. An atomizer using electrohydrodynamics can be used to produce a fine mist.

Prior to use in a dry powder or suspension formulation, the drug product can be comminuted to a particle size suitable for delivery by inhalation (e.g., 90% of the particles, based on volume, having a largest dimension less than 5 microns). This can be achieved by any appropriate size reduction method, such as spiral jet milling, fluid bed jet milling, supercritical fluid processing, high pressure homogenization, or spray drying.

Capsules, blisters, and cartridges (made, e.g., from gelatin or hydroxypropylmethyl cellulose) for use in an inhaler or insufflator can be formulated to contain a powder mixture of the active compound, a powder base such as lactose or starch, and a performance modifier, such as L-leucine, mannitol, or magnesium stearate. The lactose can be anhydrous or monohydrated. Other suitable excipients can be dextran, glucose, maltose, sorbitol, xylitol, fructose, sucrose, and trehalose. A solution formulation for use in an atomizer using electrohydrodynamics to produce a fine mist can contain from about 1 μg to about 200 mg of the API per actuation and the actuation volume may vary from about 1 μL to about 100 μL A formulation can comprise one or more compounds of Formula (I), including the compounds specifically named above, and the pharmaceutically acceptable salts thereof, propylene glycol, sterile water, EtOH, and NaCl. Other solvents can be glycerol and polyethylene glycol.

Formulations for inhaled administration, intranasal administration, or both, can be formulated to be immediate or modified release using, for example, PGLA. Flavors, such as menthol and levomenthol, or sweeteners, such as saccharin or sodium saccharin, can be added to formulations for inhaled/intranasal administration. In the case of dry powder inhalers and aerosols, the dosage unit can be determined by means of a valve that delivers a metered amount. Units can be arranged to administer a metered dose or “puff” containing from about 10 μg to about 1000 μg of the API. The overall daily dose can range from about 100 μg to about 1000 mg, and can be administered in a single dose or as divided doses throughout the day. The active compounds can be administered rectally or vaginally, e.g., in the form of a suppository, pessary, or enema. Cocoa butter can be a suppository base, but various alternatives can be used as appropriate. Formulations for rectal or vaginal administration can be formulated to be immediate or modified release as described above.

The compounds herein, and the pharmaceutically acceptable salts thereof can also be administered directly to the eye or ear, e.g., in the form of drops of a micronized suspension or solution in isotonic, pH-adjusted, sterile saline. Other formulations suitable for ocular and aural administration can include ointments, gels, biodegradable implants (e.g. absorbable gel sponges, collagen), non-biodegradable implants (e.g. silicone), wafers, lenses, and particulate or vesicular systems, such as niosomes or liposomes. The formulation can include one or more polymers and a preservative, such as benzalkonium chloride. Typical polymers can include crossed-linked polyacrylic acid, polyvinyl alcohol, hyaluronic acid, cellulosic polymers (e.g., hydroxypropylmethylcellulose, hydroxyethylcellulose, methyl cellulose), and heteropolysaccharide polymers (e.g., gelan gum). Such formulations can also be delivered by iontophoresis. Formulations for ocular or aural administration can be formulated to be immediate or modified release as described above. As noted above, the compounds herein, and the pharmaceutically acceptable salts thereof, and their pharmaceutically active complexes, solvates and hydrates, can be combined with one another or with one or more other active pharmaceutically active compounds to treat various diseases, conditions, and disorders. In such cases, the active compounds can be combined in a single dosage form as described above or can be provided in the form of a kit that can suitable for coadministration of the compositions.

The kit can comprise (1) two or more different pharmaceutical compositions, at least one of which contains a compound of Formula (I); and (2) a device for separately retaining the two pharmaceutical compositions, such as a divided bottle or a divided foil packet. A kit can be a blister pack, which can be used for the packaging of tablets or capsules. The kit can be suitable for administering different types of dosage forms (e.g., oral and parenteral) or for administering different pharmaceutical compositions at separate dosing intervals, or for titrating the different pharmaceutical compositions against one another. To assist with patient compliance, the kit can comprise directions for administration and can be provided with a memory aid.

For administration to human patients, the total daily dose of the claimed and disclosed compounds can be in the range of about 0.1 mg to about 3000 mg depending on the route of administration. For example, oral administration can be a total daily dose of from about 1 mg to about 3000 mg, while an intravenous dose can be a daily dose of from about 0.1 mg to about 300 mg. The total daily dose can be administered in single or divided doses and, at the physician's discretion, can fall outside of the typical ranges given above. Although these dosages are based on an average human subject having a mass of about 60 kg to about 70 kg, the physician can determine an appropriate dose for a patient whose mass falls outside of this weight range.

### Methods of Treatment and Uses

In some aspects, provided is a method of inhibiting fatty acid amide hydrolase (FAAH) in a subject in need thereof, comprising administering to the subject any of the compounds and compositions, including the pharmaceutical compositions, as described herein.

In certain aspects, provided is a method of treating a condition treatable by inhibition of FAAH in a subject in need thereof, comprising administering to the subject any of the compounds and compositions, including the pharmaceutical compositions, as described herein. I

In some embodiments, the methods provided treat an anxiety disorder, an insomnia disorder, a parasomnia, an obsessive-compulsive disorder, an autism spectrum disorder, a disruptive, impulse-control, and conduct (DIC) disorder, an attention-deficit/hyperactivity disorder, a trauma- and stress-related disorder, a psychotic disorder, a bipolar disorder, a depressive disorder, multiple sclerosis, spasticity, epilepsy, Niemann-Pick disease, a substance-related and addictive disorder, a neurocognitive disorder, Tourette's Syndrome, fibromyalgia, or neuropathic pain in the subject.

In certain embodiments, the method comprises administering a pharmaceutical composition comprising 3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof, and at least one pharmaceutically acceptable excipient.

In certain embodiments, the method comprises administering a pharmaceutical composition comprising (R,E)-3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof, and at least one pharmaceutically acceptable excipient.

In certain embodiments, the method comprises administering a pharmaceutical composition comprising (S,E)-3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof, and at least one pharmaceutically acceptable excipient.

In certain embodiments, the method comprises administering a pharmaceutical composition comprising (R,Z)-3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof, and at least one pharmaceutically acceptable excipient.

In certain embodiments, the method comprises administering a pharmaceutical composition comprising (S,Z)-3-hydroxy-N-(pyridazin-3-yl)-4-(3-((5-(trifluoromethyl)pyridin-2-yl)oxy)benzylidene)piperidine-1-carboxamide, or a pharmaceutically acceptable salt or solvate thereof, and at least one pharmaceutically acceptable excipient.

In certain embodiments, the method comprises administering a pharmaceutical composition comprising N-(6-oxo-1,4,5,6-tetrahydropyridazin-3-yl)-4-(3-((5-(trifluoromethyl)pyridin-2-yl)oxy)benzylidene)piperidine-1-carboxamide, or a pharmaceutically acceptable salt or solvate thereof, and at least one pharmaceutically acceptable excipient.

In certain embodiments, the method comprises administering a pharmaceutical composition comprising 4-oxo-4-(4-(3-((5-(trifluoromethyl)pyridin-2-yl)oxy)benzylidene)piperidine-1-carboxamido)butanoic acid, or a pharmaceutically acceptable salt or solvate thereof, and at least one pharmaceutically acceptable excipient.

In certain embodiments, the method comprises treating autism spectrum disorder.

In certain embodiments, the method comprises treating the trauma- and stress-related disorder. In certain embodiments, the trauma- and stress-related disorder is one or more of post-traumatic stress disorder and acute stress disorder.

In certain embodiments, the method comprises treating post-traumatic stress disorder. In certain embodiments, the method comprises a combination treatment with evidence-based psychotherapies. In other words, in some embodiments, the subject is also participating in an evidence-based psychotherapy. In certain embodiments, the evidence-based psychotherapies for PTSD and associated symptoms can be prolonged exposure (and other exposure-based techniques), cognitive processing therapy, cognitive behavioral therapy, eye movement desensitization and reprocessing therapy, skills training in affective and interpersonal regulation, concurrent treatment of PTSD and substance use disorders using prolonged exposure (and/or other exposure-based techniques), or a digital therapeutic for the treatment of PTSD or associated symptoms.

In certain embodiments, the substance-related and addictive disorder is one or more of cannabis use disorder, alcohol use disorder, hallucinogen-related disorders, opioid-related disorders, sedative-, hypnotic-, or anxiolytic-use disorder, stimulant-related disorders, and tobacco-related disorders.

In certain embodiments, the anxiety disorder is one or more of social anxiety disorder, generalized anxiety disorder, panic disorder, agoraphobia, and other specific phobias.

In certain embodiments, wherein the parasomnia is one or more of sleep-walking, nightmare disorder, REM sleep behavior disorder, and restless leg syndrome.

In certain embodiments, the psychotic disorder is one or more of schizophrenia spectrum disorder, schizophreniform disorder, schizoaffective disorder, delusional disorder, brief psychotic disorder, and attenuated psychotic disorder.

In certain embodiments, the depressive disorder is one or more of major depressive disorder, dysthymia, and premenstrual dysphoric disorder.

In certain embodiments, the neurocognitive disorder is one or more of Alzheimer's disease (AD), mild cognitive impairment (MCI), Parkinson's disease (PD), frontotemporal dementia (FTD), dementia with Lewy Bodies (DLB), progressive supranuclear palsy (PSP), corticobasal degeneration (CBD), Wernicke-Korsakoff syndrome, normal pressure hydrocephalus (NPH), prion diseases, vascular dementia, Huntington's disease (HD), and traumatic brain injury (TBI).

In certain embodiments, the subject is an adult human. In certain embodiments, the subject is a human aged 2-17 years old. In certain embodiments, the subject is a child. In certain embodiments, the subject is an adolescent.

In certain embodiments, the method comprises administering to the subject in need thereof a therapeutically effective amount of an antidepressant.

In certain embodiments, the antidepressant comprises one or more of a selective serotonin reuptake inhibitor (SSRI), a serotonin and norepinephrine reuptake inhibitor (SNRI), a norepinephrine reuptake inhibitor (NRI), or a norepinephrine and dopamine reuptake inhibitor (NDRI).

In certain embodiments, the SSRI can be one or more of sertraline, sertraline metabolite demethylsertraline, fluoxetine, norfluoxetine (fluoxetine desmethyl metabolite), fluvoxamine, paroxetine, citalopram, citalopram metabolite desmethylcitalopram, escitalopram, d,l-fenfluramine, femoxetine, ifoxetine, cyanodothiepin, litoxetine, dapoxetine, nefazodone, cericlamine, indalpine, zimelidine, and trazodone.

In certain embodiments, the SNRI can be one or more of atomoxetine, venlafaxine, venlafaxine metabolite O-desmethylvenlafaxine, clomipramine, clomipramine metabolite desmethylclomipramine, duloxetine, levomilnacipran, milnacipran, imipramine, sibutramine, and tramadol.

In certain embodiments, the NRI can be one or more of maprotiline, lofepramine, mirtazepine, oxaprotiline, fezolamine, tomoxetine, mianserin, buproprion, buproprion metabolite hydroxybuproprion, nomifensine, viloxazine, and reboxetine (including (S. S)-reboxetine).

In certain embodiments, the NDRI can be one or more of amineptine, bupropion, desoxypipradrol (2-DPMP), dexmethylphenidate, difemetorex, diphenylprolinol, ethylphenidate, fencamfamine, fencamine, lefetamine, methylenedioxypyrovalerone, methylphenidate, nomifensine, O-2172, phenylpiracetam, pipradrol, prolintane, pyrovalerone, solriamfetol, tametraline, and WY-46824.

### EXAMPLES

The compounds, pharmaceutical compositions, and methods described herein are now further detailed with reference to the following examples. These examples are provided for the purpose of illustration only and the embodiments described herein should in no way be construed as being limited to these examples. Rather, the embodiments should be construed to encompass any and all variations which become evident as a result of the teaching provided herein.

### Example 1: Synthesis of Compound A (3-hydroxy-N-(pyridazin-3-yl)-4-(3-((5-(trifluoromethyl)pyridin-2-yl)oxy)benzylidene)piperidine-1-carboxamide)

Compound A can be prepared according to scheme 1.

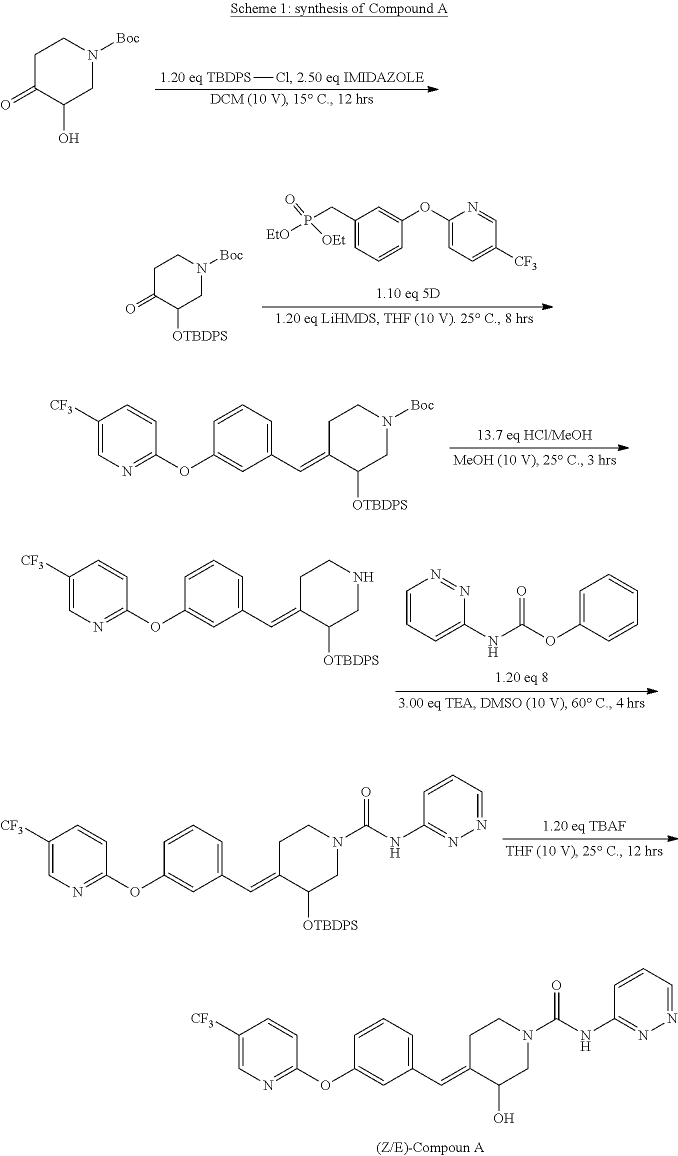

### Step 1: Preparation of Compound b (tert-butyl 3-((tert-butyldiphenylsilyl)oxy)-4-oxopiperidine-1-carboxylate)

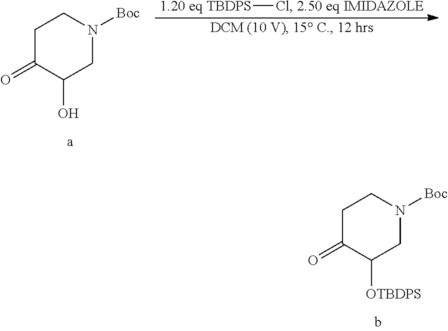

to a solution of compound a (19.0 g, 88.2 mmol, 1.00 eq) in DMF (50.0 ML) was added IMIDAZOLE (15.0 g, 220 mmol, 2.50 eq) and TBDPS-CL (29.10 g, 105.89 mmol, 27.2 mL, 1.20 eq). The mixture was stirred at 15° C. for 12 hrs. TLC (Petroleum ether:Ethyl acetate=5:1) showed that the reactant l (Rf=0.21) was consumed and a new spot (Rf=0.65) was given. The reaction mixture was poured into water (300 mL) and extracted with EtOAc (200 mL*2). The combined organic layer was washed with brine (200 mL), dried over Na₂SO₄, filtered and concentrated. The residue was purified by column chromatography (SiO₂, Petroleum ether, Petroleum ether/Ethyl acetate=15/1 to 10:1) to give a colourless oil. HNRM (EC2840-15-P1A) showed that the desired product compound b (29.0 g, crude) was obtained as colourless oil.

### Step 2: Preparation of Compound 10A (tert-butyl (E)-3-((tert-butyldiphenylsilyl)oxy)-4-(345-(trifluoromethyl)pyridin-2 yl)oxy)benzylidene)piperidine-1-carboxylate)

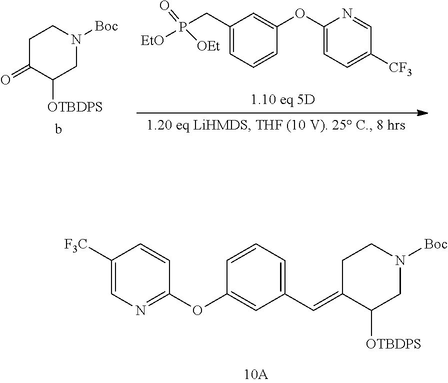

To a solution of compound b (28.0 g, 61.7 mmol, 1.00 eq) and compound 5D (25.0 g, 64.2 mmol, 1.10 eq) in THE (290 mL) at −78° C. was added LiHMDS (1.00 M, 78.0 mL, 1.20 eq). The mixture was stirred at 25° C. for 8 hrs. LCMS (EC2840-22-P1A2) showed that the main peak with desired MS (M+23=711.8, Rt=0.830 min) was found. The reaction mixture was poured into saturated NH₄C₁ aqueous solution (100 mL) and extracted with EtOAc (50.0 mL*3). The combined organic layer was washed with brine (50.0 mL), dried over Na₂SO₄, filtered and concentrated. The crude product was used next step without purification. The desired product compound 10A (52.0 g, 34.8 mmol, 54.3% yield, 46.2% purity) was obtained as yellow oil.

### Step 3: Preparation of Compound 11A ((E)-2-(343-((tert-butyldiphenylsilyl)oxy)piperidin-4-ylidene)methyl)phenoxy)-5-(trifluoromethyl)pyridine)

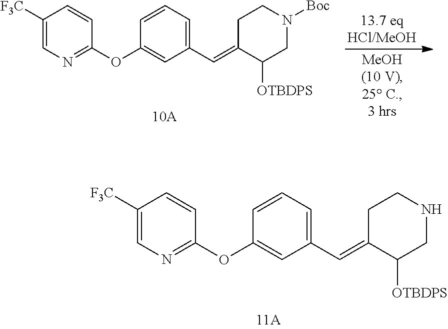

To a solution of 10A (52.0 g, 75.4 mmol, 1.00 eq) in MeOH (400 mL) was added HCl/MeOH (4.00 M, 258 mL, 13.7 eq). The mixture was stirred at 25° C. for 3 hr. LCMS (EC2840-23-P1A2) showed that the main peak with desired MS (M+1=589.2, Rt=0.635 min) was found. The reaction mixture was concentrated. The crude product was used to next step without purification. The desired product compound 11A was obtained as yellow oil.

### Step 4: Preparation of Compound 11B ((E)-3-((tert-butyldiphenylsilyl)oxy)-N-(pyridazin-3 yl)-4-(345-(trifluoromethyl)pyridin-2-yl)oxy)benzylidene)piperidine-1-carboxamide)

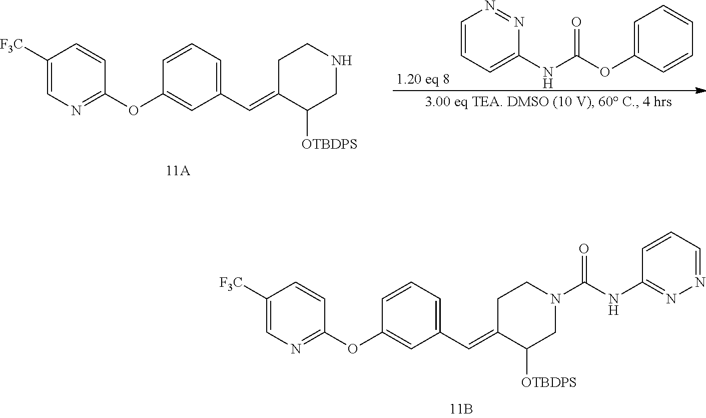

To a solution of compound 11A (50.0 g, 84.9 mmol, 1.00 eq) in DMSO (500 mL) was added TEA (26.1 g, 258.6 mmol, 36.0 mL, 3.00 eq) and compound 8 (20.0 g, 92.9 mmol, 1.20 eq) at 25° C. The mixture was stirred at 60° C. for 4 hrs. LCMS (EC2840-25-P1A2) showed that the reactant 1 was consumed and the desired MS (R t=0.755 min, M+1=710.5) was found. The reaction mixture was poured into water (200 mL) and extracted with EtOAc (100 mL*3). The combined organic layer was washed with brine (100 mL*2), dried over Na₂SO₄, filtered and concentrated. The crude product P1 (Rf=0.35) was purified by column chromatography (SiO₂, Petroleum ether/Ethyl acetate=1/0 to 5/1). The desired product compound 11B (32.0 g, crude) was obtained as yellow oil.

### Step 5: Preparation of Compound A (3-hydroxy-N-(pyridazin-3-yl)-4-(3-((5-(trifluoromethyl)pyridin-2-yl)oxy)benzylidene)piperidine-1-carboxamide)

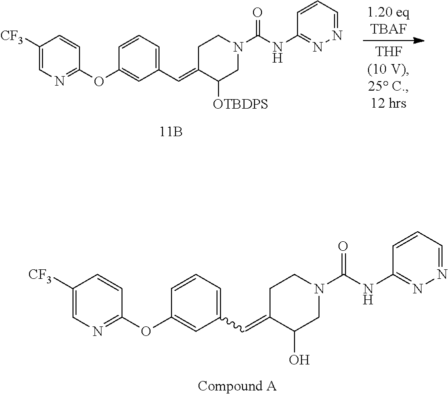

To a solution of compound 11B (32.0 g, 45.0 mmol, 1.00 eq) in THE (320 mL) was added TBAF (1.00 M, 54.0 mL, 1.20 eq) at 0° C. The mixture was stirred at 25° C. for 12 hrs. LCMS (EC2840-29-P1A) showed that the reactant 1 was consumed and the desired MS (M+1=472.1, Rt=0.515 min) was found. The reaction mixture was concentrated under reduced pressure to give a residue. Dissolve with ethyl acetate (200 mL), wash five times with saturated citric acid solution. Dry organic phase filtered and concentrated under reduced pressure to give a residue. The residue was purified by prep-HPLC (neutral condition). HPLC (EC2840-29-P1A5) showed that the desired product Compound A (9.00 g, 19.0 mmol, 42.3% yield, 99.9% purity) was obtained as white solid.

### Step 6: Chiral Supercritical Fluid Chromatography (SFC) Separation of Compound A

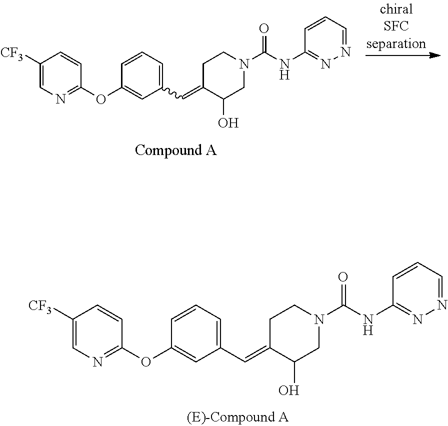 [SMILES: O=C(Nc1cccnn1)N1CCC(=Cc2cccc(Oc3ccc(C(F)(F)F)cn3)c2)C(O)C1 ; O=C(Nc1cccnn1)N1CC/C(=C\c2cccc(Oc3ccc(C(F)(F)F)cn3)c2)C(O)C1]

The product was purified by SFC (column: DAICEL CHIRALCEL OD (250 mm*50 mm, 10 um); mobile phase: [0.1% NH3H2O IPA]; B %: 55%-55%, 2.3; 320 min) to get four peak.

Single crystals of Compound A-SE were obtained from saturated solution of MTBE. Crystal data for Compound A-SE is shown in Table 1A.

TABLE 1A

| | |
|---|---|
| C₂₃H₂₀F₃N₅O₃ | F(000) = 976 |
| Mᵣ = 471.44 | Dx = 1.450 Mg m⁻³ |
| Monoclinic, P2₁ | Cu Kα radiation, λ = 1.54178 Å |
| a = 11.1794 (8) Å | Cell parameters from 9125 reflections |
| b = 5.7614 (5) Å | θ = 4.0-74.5° |
| c = 33.951 (3) Å | μ = 0.99 mm⁻¹ |
| β = 99.015 (4)° | T = 170 K |
| V = 2159.8 (3) Å³ | Block, colourless |
| Z = 4 | 0.15 × 0.08 × 0.05 mm |

Single crystals of Compound A-RE were obtained from slow evaporation from heptane/toluene (v:v=1:1). Crystal data for Compound A-RE is shown in Table 1B.

TABLE 1B

| | |
|---|---|
| C₂₃H₂₀F₃N₅O₃ | F(000) = 976 |
| Mᵣ = 471.44 | Dx = 1.448 Mg m⁻³ |
| Monoclinic, P2₁ | Cu Kα radiation, λ = 1.54178 Å |
| a = 11.1798 (6) Å | Cell parameters from 9766 reflections |
| b = 5.7680 (3) Å | θ = 2.6-74.4° |
| c = 33.9442 (19) Å | μ = 0.99 mm⁻¹ |
| β = 98.980 (3)° | T = 170 K |
| V = 2162.1 (2) Å³ | Needle, colourless |
| Z = 4 | 0.15 × 0.05 × 0.02 mm |

Single crystals of Compound A-SZ were obtained from slow evaporation from heptane/THF (v:v=2:1). Crystal data for Compound A-SZ is shown in Table 1C.

TABLE 1C

| | |
|---|---|
| C₂₃H₂₀F₃N₅O₃ | F(000) = 976 |
| Mᵣ = 471.44 | Dx = 1.438 Mg m⁻³ |
| Monoclinic, P2₁ | Cu Kα radiation, λ = 1.54178 Å |
| a = 9.3022 (2) Å | Cell parameters from 9708 reflections |
| b = 12.0294 (2) Å | θ = 2.3-75.0° |
| c = 19.4591 (4) Å | μ = 0.98 mm⁻¹ |
| β = 90.861 (1)° | T = 150 K |
| V = 2177.23 (7) Å³ | Block, colourless |
| Z = 4 | 0.11 × 0.08 × 0.06 mm |

### Example 2: Synthesis of Compound B

### Example 2-1: Synthesis of 2-(3-(piperidin-4-ylidenemethyl)phenoxy)-5-(trifluoromethyl)pyridine hydrochloride (Compound E)

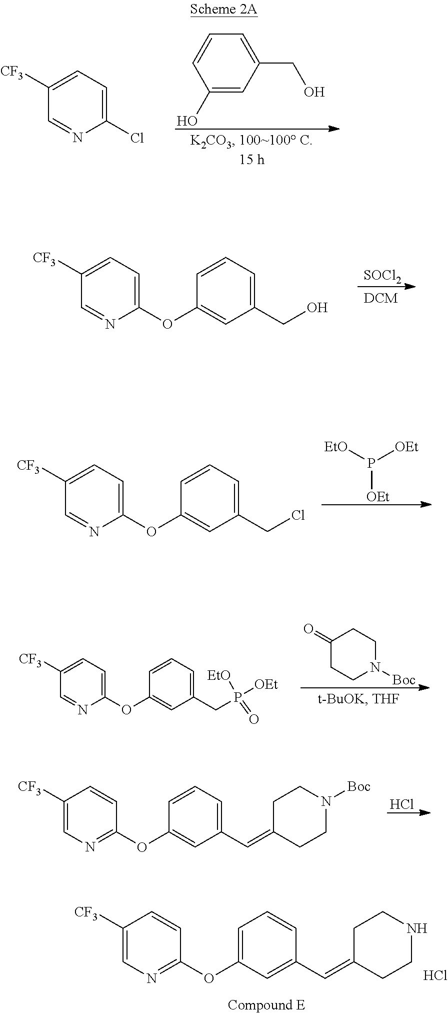

### Step 1: Preparation of (3-O-(trifluoromethyl)pyridin-2-yl)oxy)phenyOmethanol

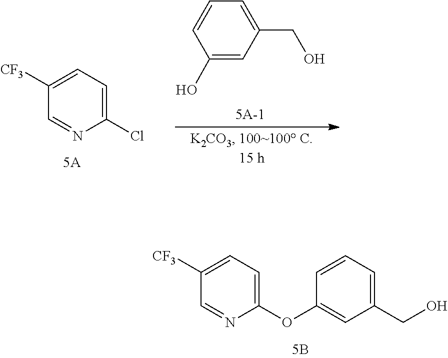 [SMILES: FC(F)(F)c1ccc(Cl)nc1 ; OCc1cccc(O)c1 ; OCc1cccc(Oc2ccc(C(F)(F)F)cn2)c1]

To a solution of compound 5A (100 g, 550.8 mmol, 70.4 mL, 1.00 eq) and K2CO3 (121 g, 881 mmol, 1.60 eq) in DMF (1.00 L) was added compound 5A-1 (75.2 g, 605 mmol, 1.10 eq) at 25° C., then the mixture was heated to 100° C. for 6 hrs. TLC (Petroleum ether:Ethyl acetate=10:1) showed reactant 1 (Rf=0.8) was consumed and a new spot (Rf=0.1) was formed. The reaction mixture was poured into water (5.00 L) and extracted with EtOAc (1.00 L*3). The combined organic layer was washed with brine (2.00 L), dried over Na₂SO₄, filtered and concentrated. The residue was used for next step without purification. From HNMR (EC1909-1-P1A), compound 5B (150 g, crude) was obtained as a yellow oil.

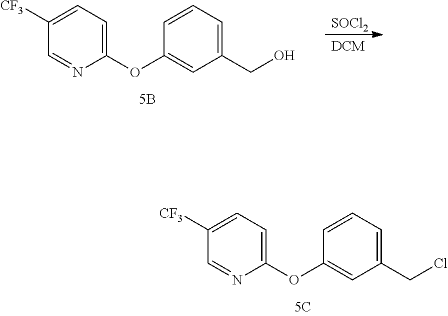 [SMILES: OCc1cccc(Oc2ccc(C(F)(F)F)cn2)c1 ; "SOCl2" ; FC(F)(F)c1ccc(Oc2cccc(CCl)c2)nc1]

### Step 2: Preparation of 2-(3-(chloromethyl)phenoxy)-5-(trifluoromethyl)pyridine

To a solution of compound 5B (120 g, 445 mmol, 1.00 eq) in DCM (600 mL) was added SOCl₂ (106 g, 891 mmol, 64.67 mL, 2.00 eq) at 0° C., then the mixture was stirred at 25° C. for 1 h. TLC (Petroleum ether:Ethyl acetate=3:1) showed reactant l (Rf=0.24) was consumed and a new spot (Rf=0.43) was formed. The reaction was concentrated. The reaction mixture was poured into saturated NaHCO₃ aqueous solution (300 mL) and extracted with DCM (300 mL*1). The combined organic layer was dried over Na₂SO₄, filtered and concentrated. The residue was used for next step without purification. From HNMR (EC1909-7-P1A), compound 5C (128 g, crude) was obtained as a yellow oil.

### Step 3: Preparation of diethyl (3((5-(trifluoromethyl)pyridin-2 yl)oxy)benzyl)phosphonate

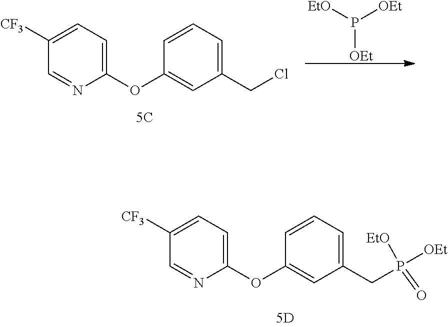 [SMILES: FC(F)(F)c1ccc(Oc2cccc(CCl)c2)nc1 ; CCOP(OCC)OCC ; CCOP(=O)(Cc1cccc(Oc2ccc(C(F)(F)F)cn2)c1)OCC]

A solution of compound 5C (120 g, 417 mmol, 1.00 eq) in triethyl phosphite (138 g, 834 mmol, 143 mL, 2.00 eq) was heated to 140° C. for 2 hrs. LCMS (EC1909-11-P1L1) showed reactant 1 was consumed and desired MS (Rt=0.558 min, m/z=390.1) was detected. The reaction solution was cooled to 15° C., n-hexane (500 mL) was added to the mixture and stirred at 15° C. for 1.0 hr. The solid was collected after filtering. The residue was used for next step without purification. From HNMR (EC1909-11-P1A), compound 5D (130 g, 333 mmol, 80.0% yield) was obtained as a Light Yellow solid.

### Step 4: Preparation of tert-butyl 4-(345-(trifluoromethyl)pyridin-2-yl)oxy)benzylidene)piperidine-1-carboxylate

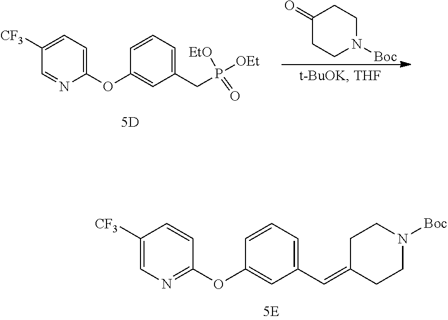 [SMILES: CCOP(=O)(Cc1cccc(Oc2ccc(C(F)(F)F)cn2)c1)OCC ; CC(C)(C)OC(=O)N1CCC(=O)CC1 ; CC(C)(C)OC(=O)N1CCC(=Cc2cccc(Oc3ccc(C(F)(F)F)cn3)c2)CC1]

To a solution of compound 5D (26.0 g, 66.80 mmol, 1.00 eq) and tert-butyl 4-oxopiperidine-1-carboxylate (13.4 g, 67.4 mmol, 1.01 eq) in THE (300 mL) at −8° C. was added t-BuOK (7.87 g, 70.1 mmol, 1.05 eq), then, the mixture was stirred at 25° C. for 12 hrs. LCMS (EC1909-33-P1A) showed 4.7% reactant 1 (Rt=0.558 min, m/z=389.9, M+1) remained and 93% desired MS (Rt=0.677 min, m.z=457, M+23) was detected. The reaction was concentrated. The mixture was added to water (200 mL) and stirred at 25° C. for 1 hr. The solid was collected after filtering. Compound 5E (28.0 g, 64.4 mmol, 96.5% yield) was obtained as a white solid.

### Step 5: Preparation of 2-(3-(piperidin-4 ylidenemethyl)phenoxy)-5-(trifluoromethyl)pyridine hydrochloride (Compound E)

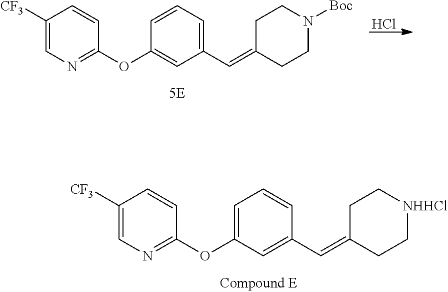

To a solution of compound 5E (15.0 g, 34.5 mmol, 1.00 eq) in EtOAc (50 mL) was added HCl/EtOAc (4 M, 70.0 mL, 8.11 eq). The mixture was stirred at 25° C. for 3 hrs. LCMS (EC1557-49-P1A3) showed that reactant 1 (m/z=434) was consumed completely and the desired MS (Rt=0.382 min, M+1=335.3) was found. The reaction mixture was concentrated under vacuum. The crude product was triturated with EtOAc (100 mL) at 25° C. for 1 hr. The mixture was filtered, and the cake was washed with EtOAc (50 mL), then the filtrate was concentrated. Compound E (9.10 g, 24.4 mmol, 70.8% yield, 99.6% purity, HCl) was obtained as white solid, checked by LCMS (EC1557-49-P1A10), HPLC (EC1557-49-P1A9), HNMR (EC1557-49-P1A1).

### Example 2-1: Synthesis of N-(6-oxo-1,4,5,6-tetrahydropyridazin-3-yl)-4-(3-((5-(trifluoromethyl)pyridin-2-yl)oxy)benzylidene)piperidine-1-carboxamide (Compound B)

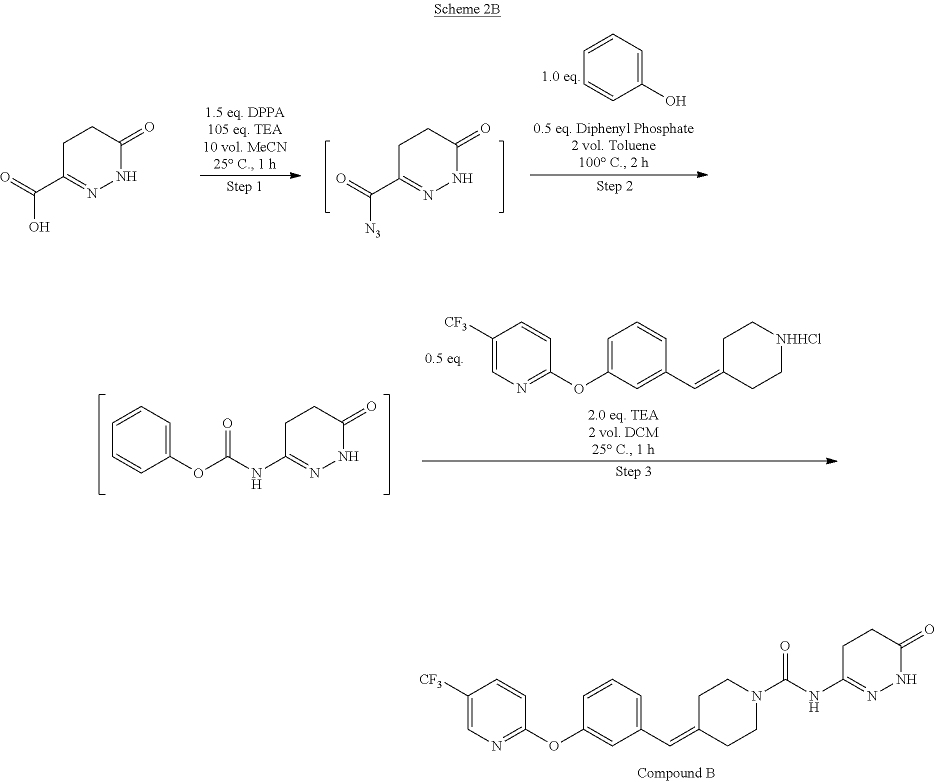

### Step 1: Preparation of 6-oxo-1,4,5,6-tetrahydropyridazine-3-carbonyl azide

To a solution 6-oxo-1,4,5,6-tetrahydropyridazine-3-carboxylic acid (1.0 eq.) and TEA (1.5 eq.) in MeCN (10 vol.), DPPA was added at 20-30° C.; over 30 minutes. The mixture was stirred 20-30° C.; for 1 hour and was used directly for the next step.

### Step 2: Preparation of phenyl (6-oxo-1,4,5,6-tetrahydropyridazin-3-yl)carbamate

To the mixture from step 1, toluene (20 vol.), diphenyl phosphate (0.5 eq.), and phenol (5 eq.) were added. The mixture was stirred at 75-80° C.; for 2 hours. The resulting reaction mixture was cooled down to 20-30° C.; and was used directly for the next step.

### Step 3: Preparation of N-(6-oxo-1,4,5,6-tetrahydropyridazin-3-yl)-4-(345-(trifluoromethyl)pyridin-2-yl)oxy)benzylidene)piperidine-1-carboxamide (Compound B)

To the mixture from step 2, toluene (0.7 eq.) and TEA (2 eq.) were added. The mixture was stirred at 20-30° C.; for 2 hours. The reaction mixture was concentrated to remove MeCN and toluene, followed by diluting with EtOAc. The organic layer was washed with 10% Na₂CO₃ solution and the resulting organic layer was washed with 1M HCl solution until pH=7. Solvent was removed to obtained crude product. Column purification to obtain Compound B.

### Example 3: Synthesis of Compound C (4-oxo-4-(4-(3-((5-(trifluoromethyl)pyridin-2-yl)oxy)benzylidene)piperidine-1-carboxamido)butanoic acid)

Compound C may be synthesized generally in accordance with scheme 3.

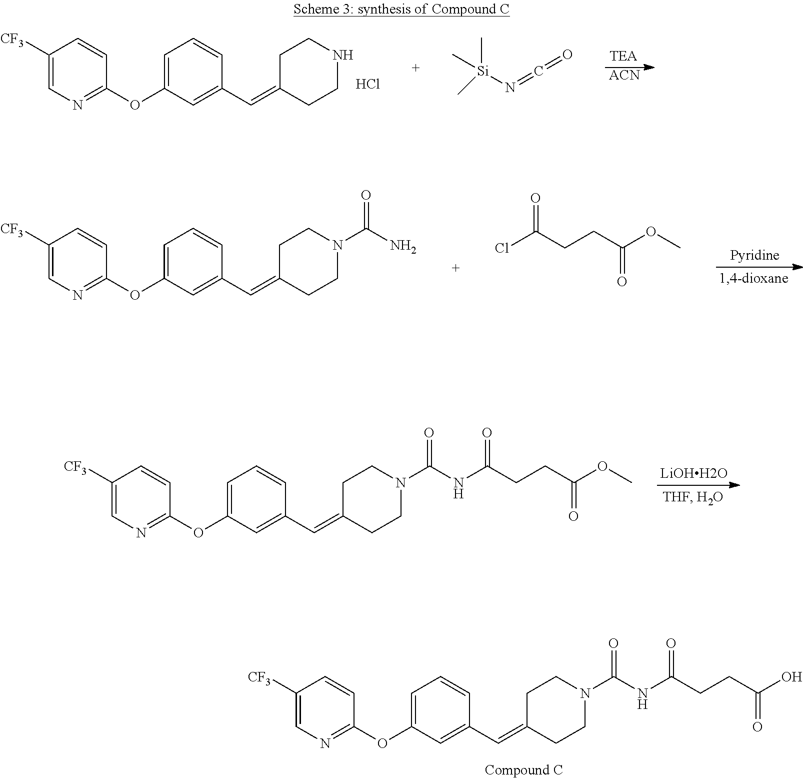

### Step 1: Preparation of 4-(3-O-(trifluoromethyl)pyridin-2 yl)oxy)benzylidene)piperidine-1-carboxamide

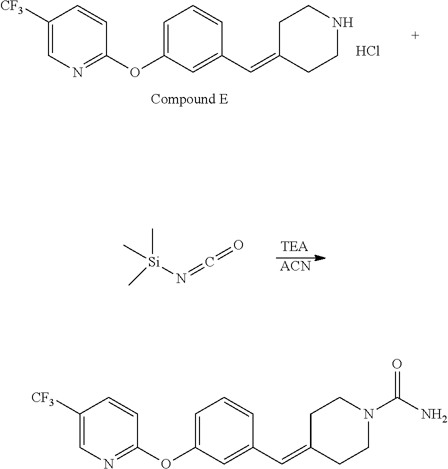

Compound E was reacted with 1.4 eq. of isocyanatotrimethylsilane in ACN and TEA at 0-10° C. for 22 hours. 4-(3-((5-(trifluoromethyl)pyridin-2-yl)oxy)benzylidene)piperidine-1-carboxamide was obtained with 99.0% LCAP in 88.2% assay yield.

### Step 2: Preparation of methyl 4-oxo-4-(4-(3-O-(trifluoromethyl)pyridin-2-yl)oxy)benzylidene)piperidine-1-carboxamido)butanoate

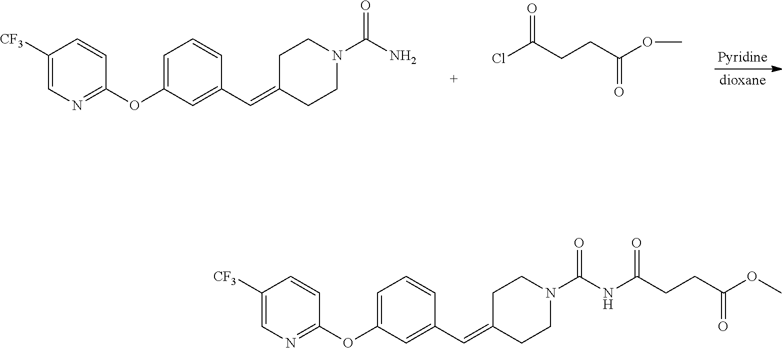

4-(3-((5-(trifluoromethyl)pyridin-2-yl)oxy)benzylidene)piperidine-1-carboxamide was reacted with 2.0 eq. of methyl 4-chloro-4-oxobutanoate and 1.5 eq. of pyridine in 1,4-dioxane at 15-25° C. for 14 hours. EtOAc and H2O were added into the reaction mixture to extract the product into EtOAc, then washed the EtOAc solution with 7% NaHCO3 and water. Methyl 4-oxo-4-(4-(3-((5-(trifluoromethyl)pyridin-2-yl)oxy)benzylidene)piperidine-1-carboxamido)butanoate was purified by column chromatography, with 98.6% LCAP in 64.4% assay yield.

### Step 3: Preparation of 4-oxo-4-(4-(345-(trifluoromethyl)pyridin-2-yl)oxy)benzylidene)piperidine-1-carboxamido)butanoic acid (Compound C)

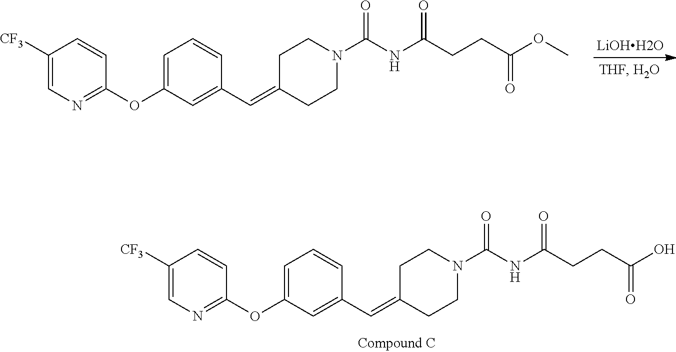 [SMILES: COC(=O)CCC(=O)NC(=O)N1CCC(=Cc2cccc(Oc3ccc(C(F)(F)F)cn3)c2)CC1 ; O=C(O)CCC(=O)NC(=O)N1CCC(=Cc2cccc(Oc3ccc(C(F)(F)F)cn3)c2)CC1]

Methyl 4-oxo-4-(4-(3-((5-(trifluoromethyl)pyridin-2-yl)oxy)benzylidene)piperidine-1-carboxamido)butanoate was reacted with 2.2 eq. of LiOH·H2O in THE at 0-5° C. for 15 hours. Aqueous layer of 4-oxo-4-(4-(3-((5-(trifluoromethyl)pyridin-2-yl)oxy)benzylidene)piperidine-1-carboxamido)butanoic acid lithium salt was separated and washed with EtOAc, followed by adjusting the aqueous layer pH to 3-4 for crystallization. 4-oxo-4-(4-(3-((5-(trifluoromethyl)pyridin-2-yl)oxy)benzylidene)piperidine-1-carboxamido)butanoic acid was obtained with 99.0% LCAP in 79.5% assay yield.

### Example 4: Human FAAH Activity

Human FAAH protein was prepared using the following method: Recombinant human FAAH encoding full length amino acids were expressed in insect cells using baculovirus infection as C-terminal (non-cleavable) 8×His tag fusion protein. Protein was supplied as cell lysate: resuspended 100,000×g pellet at 5.12 mg/ml a buffer containing 20 mM HEPES, pH 7.8, 150 mM NaCl, 1 mM EDTA, 1 mM DTT, 0.5% CHAPS. Specific activity was determined to be 844 U/mg.

Fluorometric-based Human FAAH Inhibition Assay was performed using the following method:

Assays were performed in standard-volume, black, non-binding 384-well plates (Corning #3654). All additions were carried out using the Agilent Bravo Automated Liquid Handler using an 384ST pipetting head.

FAAH reactions contained 125 nM Fatty Acid Amide Hydrolase (human, recombinant) diluted in an assay buffer containing Tris-EDTA, pH 9.0 at ambient temperature. To obtain 125 nM FAAH per well, a working stock was made at 312.5 nM by diluting 94 μL of 79.63 μM enzyme to 24 mL of assay buffer, then adding 20 μL to each well. Using the mother plates (prepared at 1000× in DMSO), the Agilent Bravo performed a 1:100 dilution step in assay buffer prior to adding 5 μL to each well for an additional 1:10 dilution.

Plates were incubated at room-temperature for 1 hour.

To initialize the reaction, a final concentration of 204 of AMC Arachidonoyl Amide was added to each well. To obtain this concentration, a working stock was made at 404 by adding 960 μL of 0.1 mM AMC stock to 24 mL of assay buffer, then 25 μL was added to each well. This stock was made at 4% DMSO for a final assay concentration contribution of 2% DMSO.

The total reaction volume per well was 50 μL. The reactions were allowed to proceed at 37° C. for 30 minutes with fluorescence monitored using a Cytation 5 Multimode imaging plate reader (BioTek Instruments) using excitation and emission wavelengths of 350/10 nm and 455/10 nm, respectively. Rates (RFU/min) were calculated from the linear portion of the reaction curves using a linear least squares curve fitting method using Gen5 v3.10. Calculated rates were then normalized to DMSO control and presented as percentages. Data were graphed and statistics were performed using GraphPad Prism v9.0.1.

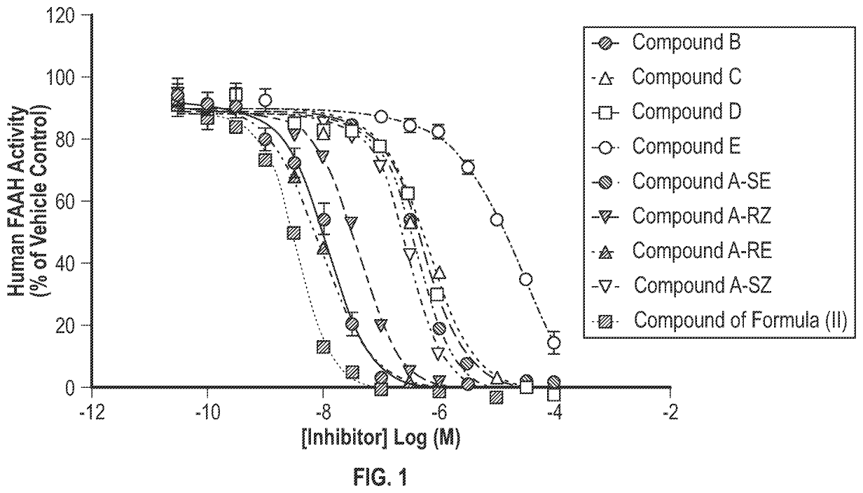

Data for the Fluorometric-based Human FAAH Inhibition Assay are recited in Table 2 and in FIG. 1.

### Example 5: Rat FAAH Activity

Rat FAAH protein was prepared using the following method: Recombinant truncated rat FAAH encoding amino acids residues 30-579 were expressed in E. coli as NH2-terminal 6×HIS-tag and a TEV-cleavage site fusion protein. 6 L pLysS cells were induced to express FAAH. Cells were lysed and centrifuged. The insoluble pellet was resuspended using a Dounce homogenizer in CHAPS-containing buffer and allowed to stir at 4° C. for 1 hour. The resuspended pellet was then centrifuged and rat FAAH in the clarified supernatant was purified by IMAC. The purest protein fractions were pooled, and buffer exchanged into storage buffer by dialysis. The protein concentration was determined by Bradford assay aliquots were flash frozen in liquid nitrogen.

Fluorometric-based Rat FAAH Inhibition Assay was performed using the following method:

Assays were performed in standard-volume, black, non-binding 384-well plates (Corning #3654). All additions were carried out using the Agilent Bravo Automated Liquid Handler using an 384ST pipetting head.

FAAH reactions contained 20 nM Fatty Acid Amide Hydrolase (rat, recombinant) diluted in an assay buffer containing Tris-EDTA, pH 9.0 at ambient temperature. To obtain 20 nM FAAH per well, a working stock was made at 50 nM by diluting 150 μL of 7.93 μM enzyme to 24 mL of assay buffer, then adding 20 μL to each well. Using the mother plates (prepared at 1000× in DMSO), the Agilent Bravo performed a 1:100 dilution step in assay buffer prior to adding 5 μL to each well for an additional 1:10 dilution.

Plates were incubated at room temperature for 1 hour.

To initialize the reaction a final concentration of 2 μM of AMC Arachidonoyl Amide was added to each well. To obtain this concentration a working stock was made at 4 μM by adding 960 μL of 0.1 mM AMC stock to 24 mL of assay buffer, then 25 μL was added to each well. This stock was made at 4% DMSO for a final assay concentration contribution of 2% DMSO.

The total reaction volume per well was 50 μL. The reactions were allowed to proceed at 37° C. for 30 minutes with fluorescence monitored using a Cytation 5 Multimode imaging plate reader (BioTek Instruments) using excitation and emission wavelengths of 350/10 nm and 455/10 nm, respectively. Rates (RFU/min) were calculated from the linear portion of the reaction curves using a linear least squares curve fitting method using Gen5 v3.10. Calculated rates were then normalized to DMSO control and presented as percentages. Data were graphed and statistics were performed using GraphPad Prism v9.0.1.

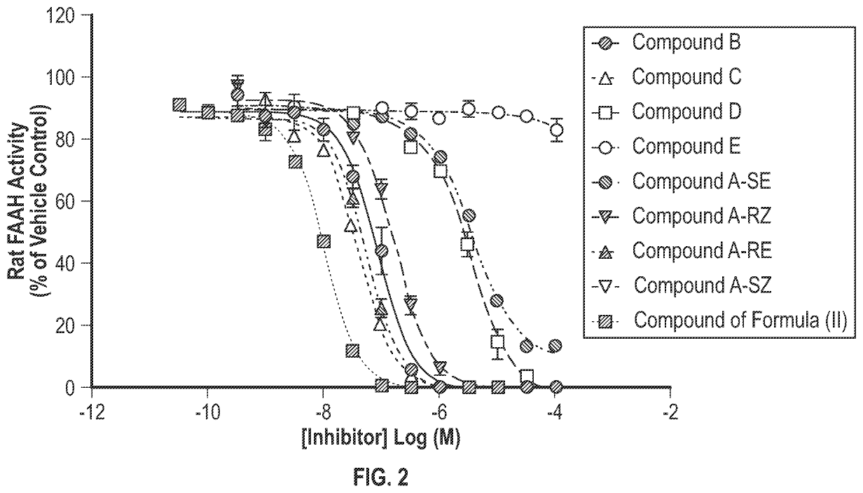

Data for the Fluorometric-based Rat FAAH Inhibition Assay are recited in Table 2 and in FIG. 2.

TABLE 2
Human FAAH and Rat FAAH ICso Data
for Compounds of the Disclosure

| | Human FAAH | Rat FAAH |
|---|---|---|
| | IC₅₀ (nM) | IC₅₀ (nM) |
| Compound B | 12.4 ± 0.89 | 85.2 ± 9.334 |
| Compound C | 758 ± 11.2 | 39.6 ± 5.84 |
| Compound D | 555 ± 84.4 | 3481 ± 654 |
| Compound E | 34400 ± 24100 | N/A |
| Compound A-SE | 439 ± 14.7 | 3720 ± 542 |
| Compound A-RZ | 39.3 ± 2.89 | 170 ± 43 |
| Compound A-RE | 9.01 ± 0.68 | 56.8 ± 16.6 |
| Compound A-SZ | 300.20 ± 54.9 | 3290 ± 583 |
| Compound of Formula (II) | 3.56 ± 0.67 | 10.2 ± 1.17 |

Compound of Formula (II) has the following structure:

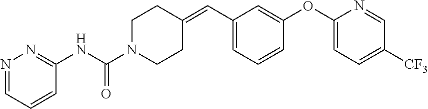 [SMILES: O=C(Nc1cccnn1)N1CCC(=Cc2cccc(Oc3ccc(C(F)(F)F)cn3)c2)CC1]

Compound D (4-hydroxy-4-(hydroxy(3-((5-(trifluoromethyl)pyridin-2-yl)oxy)phenyl)methyl)-N-(pyridazin-3-yl)piperidine-1-carboxamide) has the following structure:

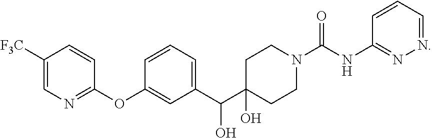 [SMILES: O=C(Nc1cccnn1)N1CCC(O)(C(O)c2cccc(Oc3ccc(C(F)(F)F)cn3)c2)CC1]

Compound E (2-(3-(piperidin-4-ylidenemethyl)phenoxy)-5-(trifluoromethyl)pyridine) has the following structure:

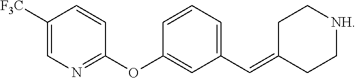 [SMILES: FC(F)(F)c1ccc(Oc2cccc(C=C3CCNCC3)c2)nc1]

Examples 4 and 5 thus demonstrate that a compound of Formula (I) can be effective for inhibiting FAAH.

Examples 4 and 5 thus surprisingly demonstrate that a compound of Formula (I) can inhibit FAAH.

### Example 6: Pharmacokinetic Study

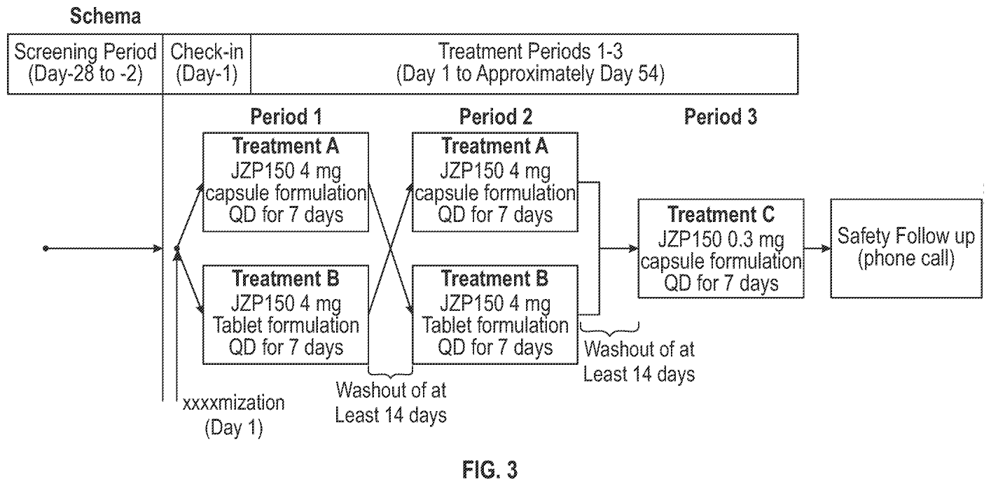

Partial preliminary data on Compound of Formula (II), Compound B, and Compound C) were obtained from a phase 1, single-center, open-label, randomized, 3-period, 2-sequence crossover study to characterize the PK, safety, and tolerability of Compound of Formula (II) formulations (capsule vs tablet) in healthy adult participants. In Periods 1 and 2, participants were randomized to receive 4 mg Compound of Formula (II) capsule formulation or 4 mg Compound of Formula (II) tablet formulation. Study schema is as shown in FIG. 3.

Pharmacokinetic parameters for compound of Formula (II), Compound B, and Compound C following single dose of 4 mg of compound of Formula (II) are provided in Table 3. Pharmacokinetic parameters for compound of Formula (II), Compound B, and Compound C following multiple doses of 4 mg of compound of Formula (II) are provided in Table 4.

TABLE 3

| PK | | Compound of | | |
|---|---|---|---|---|
| parameter | Units | Formula (II) | Compound B | Compound C |
| Cₘₐₓ | ng/ml | 31.3 (34.7) | 0.3 (33.1) | 0.4 (39.2) |
| AUCₗₐₛₜ | ng*h/mL | 137.8 (26.2) | 0.6 (33.1) | 1.1 (47.5) |

TABLE 4

| PK | | Compound of | | | | | |
|---|---|---|---|---|---|---|---|
| parameter | Units | Formula (II) | | Compound B | | Compound C | |
| Cₘₐₓ | ng/ml | 46.6 | (26.0) | 9.3 | (37.8) | 3.9 | (25.2) |
| Tₘₐₓ | h | 2.0 | (1.0-4.0) | 24.0 | (1.0-24.0) | 8.0 | (0.5-24.0) |
| AUCₜₐᵤ | ng*h/mL | 624.6 | (24.3) | 207.9 | (39.5) | 86.0 | (24.8) |
| AUCₗₐₛₜ | ng*h/mL | 1338.6 | (26.6) | 905.8 | (38.5) | 280.7 | (29.5) |
| _{T1/2} | h | 32.2 | (18.7) | 85.0 | (38.4) | 40.4 | (21.3) |

1. A pharmaceutical composition comprising:
a compound of Formula (I) or a pharmaceutically acceptable salt or solvate thereof; and at least one pharmaceutically acceptable excipient,
wherein Formula (I) is: 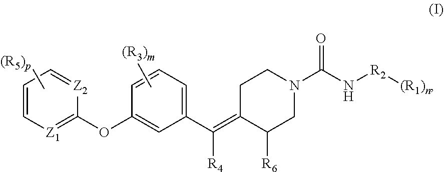
wherein:
each R₁ is independently hydrogen, —C₁-C₆ alkyl, —OH, or —O—(C₁-C₆ alkyl);
R₂ is —C(O)—C₁-C₆ alkyl-C(O)—R₇, a 6-membered aromatic heterocycle containing 1 or 2 nitrogen ring heteroatoms, or dihydropyridazinone, wherein R₇ is hydrogen, halogen, —C₁-C₆ alkyl, —OH, —O—C₁-C₆ alkyl, or —O-haloalkyl;
R₆ is hydrogen or —OH;
each R₃ is independently hydrogen, halogen, —C₁-C₆ alkyl, —(CH₂)₀₋₃—C₃-C₆ cycloalkyl, or —O—C₁-C₆ alkyl;
R₄ is hydrogen, —OH, —C₁-C₆ alkyl, phenyl, or halogen;
each R₅ is independently hydrogen, halogen, haloalkyl, —O-haloalkyl, —C₁-C₆ alkyl, —C(O)C₁-C₆ alkyl, —O—C₁-C₆ alkyl, —S—C₁-C₆ alkyl, —(CH₂)₀₋₃—C₃-C₆ cycloalkyl, CN, aryl, and heteroaryl; wherein the —C₁-C₆ alkyl, —O(C₁-C₆ alkyl), —(CH₂)₀₋₃—C₃-C₆ cycloalkyl, aryl, and heteroaryl groups being optionally independently substituted with from 1 to 4 —C₁-C₆ alkyl, —OH, or halogen substituents;
m is 0, 1, 2, 3, or 4;
n is 0, 1, 2, 3, or 4;
p is 0, 1, 2, 3, or 4; and
Z₁ and Z₂ are independently selected from N and CH,
with the proviso that when R₂ is a 6-membered aromatic heterocycle containing 1 or 2 nitrogen ring heteroatoms, R₆ is not H.

2. The pharmaceutical composition according to claim 1, wherein Z₁ is N and Z₂ is CH.

3. The pharmaceutical composition according to claim 1, wherein R₅ is CF₃.

4. The pharmaceutical composition according to claim 1, wherein Z₁ is N, Z₂ is CH, p is 1, and R₅ is CF₃.

5. The pharmaceutical composition according to claim 1, wherein R₆ is —OH.

6. The pharmaceutical composition according to claim 1, wherein R₂ is pyridazine.

7. The pharmaceutical composition according to claim 1, wherein R₆ is H and R₂ is —C(O)—C₁-C₆ alkyl-C(O)—R₇ or dihydropyridazinone.

8. The pharmaceutical composition according to claim 1, wherein n is 0.

9. The pharmaceutical composition according to claim 1, wherein the compound of Formula (I) is: 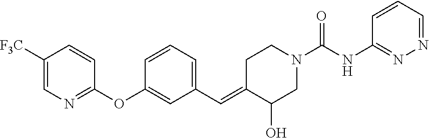 [SMILES: O=C(Nc1cccnn1)N1CC/C(=C\c2cccc(Oc3ccc(C(F)(F)F)cn3)c2)C(O)C1]
or a pharmaceutically acceptable salt or solvate thereof.

10. The pharmaceutical composition according to claim 1, wherein the compound of Formula (I) is:
(R,E)-3-hydroxy-N-(pyridazin-3-yl)-4-(3-((5-(trifluoromethyl)pyridin-2-yl)oxy)benzylidene)piperidine-1-carboxamide;
(S,E)-3-hydroxy-N-(pyridazin-3-yl)-4-(3-((5-(trifluoromethyl)pyridin-2-yl)oxy)benzylidene)piperidine-1-carboxamide;
(R,Z)-3-hydroxy-N-(pyridazin-3-yl)-4-(3-((5-(trifluoromethyl)pyridin-2-yl)oxy)benzylidene)piperidine-1-carboxamide; or
(S,Z)-3-hydroxy-N-(pyridazin-3-yl)-4-(3-((5-(trifluoromethyl)pyridin-2-yl)oxy)benzylidene)piperidine-1-carboxamide,
or a pharmaceutically acceptable salt or solvate thereof.

11. The pharmaceutical composition according to claim 1, wherein the compound of Formula (I) is: 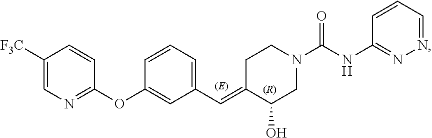
or a pharmaceutically acceptable salt or solvate thereof.

12. The pharmaceutical composition according to claim 1, wherein the compound of Formula (I) is: 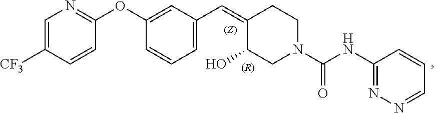
or a pharmaceutically acceptable salt or solvate thereof.

13. The pharmaceutical composition according to claim 1, wherein the compound of Formula (I) is: 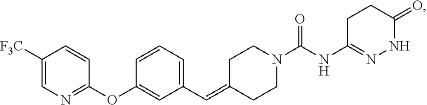 [SMILES: O=C1CCC(NC(=O)N2CCC(=Cc3cccc(Oc4ccc(C(F)(F)F)cn4)c3)CC2)=NN1]
or a pharmaceutically acceptable salt or solvate thereof.

14. The pharmaceutical composition according to claim 1, wherein the compound of Formula (I) is: 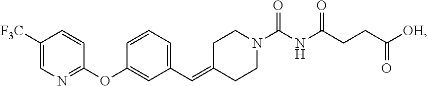 [SMILES: O=C(O)CCC(=O)NC(=O)N1CCC(=Cc2cccc(Oc3ccc(C(F)(F)F)cn3)c2)CC1]
or a pharmaceutically acceptable salt or solvate thereof.

15. The pharmaceutical composition of claim 1, wherein the pharmaceutical composition comprises the compound of Formula (I), or a pharmaceutically acceptable salt thereof.

16. The pharmaceutical composition of claim 1, wherein the pharmaceutical composition comprises a solid oral dosage form.

17. The pharmaceutical composition of claim 1, wherein the pharmaceutical composition is a tablet.

18. A method of inhibiting fatty acid amide hydrolase (FAAH) in a subject in need thereof, comprising administering to the subject a pharmaceutical composition according to claim 1.

19. A method of treating an autism spectrum disorder or post-traumatic stress disorder in a subject in need thereof, comprising administering to the subject a pharmaceutical composition according to claim 1.

20.-33. (canceled)
